SEQUENCE LISTING (12 sequences)

SEQ ID NO: 1 - DNA, 1647 bp, Arabidopsis thaliana
ataaaagtctgtggcgaccaaaagaagtgagagagaaagagagagatatgatattattaagtagtagtataagcctctcaagaccagtttcttctcaaagcttctctccacctgctgccacttcaacaaggtatggttttatgttttgaatcacaacttcattagctccgattaacctcaagatcattaatttagttataggtttaggaacaaagttacagaatcagatcctggaatggaactatcttggattatagagattcttggtttactgatctcagaaattctgttttctcttggtttcacttatgacaggagatctcattcctctgtcacagtcaagtgctgctgttcttccagaaggttgttgaagaatcctgagttaaaatgttccttggagaatctctttgaaatccaggctttgaggaagtgttttgtttcagggtttgcagctattttgcttctctctcaggcaggccaggtatgttggagccgacattgctataagcatgttcgattgcaaccacaagcggtctgaaatttctgagtttcttctggcagggtatagcgttggatctctcatctggttatcagaacatttgccaactagggagtgctgctgctgtgggagaaaacaagctgactcttccatctgatggtgactcggaatcaatgatgatgatgatgatgagaggcatgactgctaagaactttgaccctgttaggtactctggaagatggtttgaagtagcttctcttaagcgtggatttgcaggtcaaggccaagaagactgtcattgcactcaggtaattgtttcttcaagtccacaaattgtgaaaacacatcctctgctaacatagtgtgagaacctttcaactgttgcagggagtatacacgtttgatatgaaggaatcagccattagagtagatacattttgtgttcatggcagccctgatggatatataacaggaatcagagggaaagttcaatgcgtgggagcggaagacctcgagaaaagcgagactgacttagaaaagcaagagatgattaaagagaagtgtttcctacgatttcccaccattccttttatccccaagttgccttatgatgtcatagccacagactacgacaactacgcacttgtttctggagccaaagacaagggctttgttcaggtgattctaagagaaagaactcatgagtttgagaaatatctcttgcactgatctcttatataaacctaatgtgtttgatatgttaggtatactcaaggacgccaaatccaggacctgagttcatcgcaaagtacaagaactacttggcacaatttggctatgacccggaaaaaataaaggatacaccacaggactgtgaagtgactgatgctgagctagcagccatgatgtccatgccaggtatggagcaaacactgaccaaccagtttccagatcttggattaagaaagtcagtccagtttgatcccttcacaagtgtgtttgaaaccttgaagaaacttgtaccgctctatttcaaatagagcaagcttctttgctcaaattcttatgtagactataatcactgtccatatatacatatcttccagaatcaaaacactcttctgatcataacaccattgattagagaa

SEQ ID NO: 2 - protein, 353 aa, Arabidopsis thaliana
MILLSSSISLSRPVSSQSFSPPAATSTRRSHSSVTVKCCCSSRRLLKNPELKCSLENLFEIQALRKCFVSGFAAILLLSQAGQGIALDLSSGYQNICQLGSAAAVGENKLTLPSDGDSESMMMMMMRGMTAKNFDPVRYSGRWFEVASLKRGFAGQGQEDCHCTQGVYTFDMKESAIRVDTFCVHGSPDGYITGIRGKVQCVGAEDLEKSETDLEKQEMIKEKCFLRFPTIPFIPKLPYDVIATDYDNYALVSGAKDKGFVQVYSRTPNPGPEFIAKYKNYLAQFGYDPEKIKDTPQDCEVTDAELAAMMSMPGMEQTLTNQFPDLGLRKSVQFDPFTSVFETLKKLVPLYFK

SEQ ID NO: 3 - DNA, 1987 bp, Arabidopsis thaliana
aaattaaataatggcttctccttaaaattatctaagctcgcttgaccaatcatggctaccaccaccaatctcagcttcgctcctccctcttactcccgttttgccgctacaaaatcccaaatcagaaaccctctgtttacgtcccccctgccactcccatcttctttcttcctggttagaaatgaagcatctttatcttcgtcaatcacgccagtccaagcttttacagaagaagccgtcgatacttcggatttagcttcttcttcttcaaagcttgtacttgtcgttggcggcactggtggtgtaggtacttaaatgttcaattttgaaattaggactatgaatttctcactattgggtctgctgaatgatggttgttttaaagtcttatcctttttggtatcaggtcaacttgtggtagcttcattgctcaagaggaatataagatcaaggttattactgcgtgatcttgacaaagctaccaagttatttggcaaacaagatgaatattccttgcaggttcggacttaatctcactatttcggatacactacttgttctttttatacgataagtttaaaccatgttgttataggtagttaaaggggatactaggaatgcagaggatcttgatccatccatgtttgaggttcgtcttcttgctcctgttttgtttggcacgagtttcacttgttcttcatttgatttttaactgatttgcctagggtgtcacacatgtgatttgtaccactggaactacagcttttccttctaagaggtggaatgaagaaaacactcctgagaaagtaggtgagaacacatttctttccaattaagaacgttagatgtcttgttttgttatcagttctttcttacatgtcatgatcgattttcctttttgggactatgtgtttttttttgtgttcaaaaagattgggaaggtgtgaagaatctcatttcagcattgccatcatcggtgaagagagttgttttggtttcatcagtaggtgtgaccaagtctaatgagctaccctggaggtgagactagagtatctccgctaatttactttagtctgattgtctctacgtgtagtttatttgctgaatgtatgttttgtttgacagcatcatgaacctttttggagttcttaagtacaagaagatgggggaagattttcttcgtgactctggtcttccattcaccattatcaggtttcgaacaaaagagttacccactttttttgctcttaatactccaaaacgcaggcaacatatggtttactgatttactgtaatgtgatgttttcttaccagacctggtagattgactgatggaccatacacatcttatgatctgaatactttgctcaaagctacagctggtgaaaggcgtgcagttgttattggtcaaggtaaatttgagattctttgatggtcttgcagcactaagatttttgagattactttatttacataatggctccttttaacaggggacaaccttgttggagaggtaagtagacttgtagtggctgaagcttgtatacaggcacttgatattgaattcacacaaggcaaagcttacgagatcaattcagtaaaggtaccacaaacagtttccttgaaatttgaacaaagtggaaatgtagtctgatcaagaaacaatgtcttaaggggtgtcctggagattcaaagctttagttgatcattgaaaatgtttcaaagatttggttgccatttttttatatgtttgagaaatgtggtttcagggggatggtccaggaagtgatccacagcaatggcgagagttgtttaaagctgcagaatccaaatgacaaaagaggacttttgagagatgtgtacagaattgttagcgagacattacatatatggtcgattgtgtatacatgtgctttcttttggtctttgacttcatcattactgtaattactttatctataactagaagttctttcttgcaaatcaa

SEQ ID NO: 4 - protein, 338 aa, Arabidopsis thaliana
MATTTNLSFAPPSYSRFAATKSQIRNPLFTSPLPLPSSFFLVRNEASLSSSITPVQAFTEEAVDTSDLASSSSKLVLVVGGTGGVGQLVVASLLKRNIRSRLLLRDLDKATKLFGKQDEYSLQVVKGDTRNAEDLDPSMFEGVTHVICTTGTTAFPSKRWNEENTPEKVDWEGVKNLISALPSSVKRVVLVSSVGVTKSNELPWSIMNLFGVLKYKKMGEDFLRDSGLPFTIIRPGRLTDGPYTSYDLNTLLKATAGERRAVVIGQGDNLVGEVSRLVVAEACIQALDIEFTQGKAYEINSVKVPQTVSLKFEQSGNGDGPGSDPQQWRELFKAAESK

SEQ ID NO: 5 - DNA, 10988 bp, Artificial Sequence
  The nucleotide sequence of the pEarleyGate 100 expression vector comprising a 35S promoter-OTK1cDNA-Flag tag
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagatcatggctaccaccaccaatctcagcttcgctcctccctcttactcccgttttgccgctacaaaatcccaaatcagaaaccctctgtttacgtcccccctgccactcccatcttctttcttcctggttagaaatgaagcatctttatcttcgtcaatcacgccagtccaagcttttacagaagaagccgtcgatacttcggatttagcttcttcttcttcaaagcttgtacttgtcgttggcggcactggtggtgtaggtcaacttgtggtagcttcattgctcaagaggaatataagatcaaggttattactgcgtgatcttgacaaagctaccaagttatttggcaaacaagatgaatattccttgcaggtagttaaaggggatactaggaatgcagaggatcttgatccatccatgtttgagggtgtcacacatgtgatttgtaccactggaactacagcttttccttctaagaggtggaatgaagaaaacactcctgagaaagtagattgggaaggtgtgaagaatctcatttcagcattgccatcatcggtgaagagagttgttttggtttcatcagtaggtgtgaccaagtctaatgagctaccctggagcatcatgaacctttttggagttcttaagtacaagaagatgggggaagattttcttcgtgactctggtcttccattcaccattatcagacctggtagattgactgatggaccatacacatcttatgatctgaatactttgctcaaagctacagctggtgaaaggcgtgcagttgttattggtcaaggggacaaccttgttggagaggtaagtagacttgtagtggctgaagcttgtatacaggcacttgatattgaattcacacaaggcaaagcttacgagatcaattcagtaaaggtaccacaaacagtttccttgaaatttgaacaaagtggaaatggggatggtccaggaagtgatccacagcaatggcgagagttgtttaaagctgcagaatccaaatgagactataaggatgatgatgataagtaggcctaggtgagtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 6 - DNA, 20 bp, Artificial Sequence
  Primer MDB74 forward
taggtgtgcctaccagcgag

SEQ ID NO: 7 - DNA, 21 bp, Artificial Sequence
  Primer MDB72 reverse
tgagccaccagtgagaatgtc

SEQ ID NO: 8 - DNA, 28 bp, Artificial Sequence
  Primer AM145 forward
gaagtggtttcttttgtacaattctgca

SEQ ID NO: 9 - DNA, 20 bp, Artificial Sequence
  Primer AM146 reverse
caatacgaatagcgcacacg

SEQ ID NO: 10 - DNA, 22 bp, Artificial Sequence
  Primer AM164 forward (LP)
ccgctttgacatttacattacg

SEQ ID NO: 11 - DNA, 21 bp, Artificial Sequence
  Primer AM165 reverse (RP)
tatagcaatgtcggctccaac

SEQ ID NO: 12 - protein, 18 aa, Arabidopsis thaliana
TVTPRAPDAGGLQLQGTR

(19) United States
(12) Patent Application Publication
(10) Pub. No.: US 2019/0292556 A1
(43) Pub. Date: Sep. 26, 2019

(54) METHODS AND COMPOSITIONS FOR IMPROVING PHOTOSYNTHESIS

(71) Applicant: The Regents of the University of California, Oakland, CA (US)

(72) Inventors: Alizee Malnoe, Berkeley, CA (US); Krishna K. Niyogi, Lafayette, CA (US)

(73) Assignee: The Regents of the University of California, Oakland, CA (US)

(21) Appl. No.: 16/269,371

(22) Filed: Feb. 6, 2019

Related U.S. Application Data
(63) Continuation of application No. PCT/US2017/045686, filed on Aug. 7, 2017.
(60) Provisional application No. 62/371,741, filed on Aug. 6, 2016.

(51) Int. Cl. C12N 15/82; C07K 14/415

(52) U.S. Cl. CPC C12N 15/8269; C12N 15/8218; C07K 14/415

(57) ABSTRACT

Methods and compositions for improving photosynthesis by eliminating a sustained photoprotective mechanism by mutating or silencing the Chloroplastic Lipocalin (CHL) gene whereby photosynthesis of the plants increases. The sustained photoprotective mechanism negatively regulated by the Suppressor of Quenching 1 protein involves the chloroplastic lipocalin and occurs in the peripheral antenna of photosystem II.

### RELATED PATENT APPLICATIONS

The application claims priority as a continuation application to PCT International Patent Application No. PCT/US2017/045686, filed Aug. 7, 2017, which claims priority to U.S. Provisional Patent Application Ser. No. 62/371,741, filed Aug. 6, 2016, both of which are herein incorporated by reference in their entireties.

### STATEMENT OF GOVERNMENT SUPPORT

This invention was made with Government support under Contract No. DE-AC02-05CH11231 awarded by the Department of Energy. The Government has certain rights in the invention.

### FIELD OF THE INVENTION

The present invention relates to methods and compositions for improving photosynthesis by eliminating a sustained photoprotective mechanism using negative regulation of proteins.

### BACKGROUND OF THE INVENTION

Photosynthetic organisms experience various abiotic stresses that can lead to cellular damage (Li et al., 2009b). To cope with excess light, they have evolved photoprotective mechanisms that safely dissipate excess absorbed light energy as heat (Horton et al., 1996). These mechanisms are commonly called non-photochemical quenching (NPQ) as opposed to photochemical quenching, which reflects photochemistry, the process in which light energy is converted to chemical energy in the form of ATP and NADPH. The term “quenching” originates from the way these processes are assayed through monitoring a decrease (i.e., quenching) of chlorophyll fluorescence.

NPQ mechanisms have been classified according to their relaxation kinetics and their sensitivities to chemical inhibitors and mutations (Walters and Horton 1993, Nilkens et al., 2010). Energy-dependent quenching, qE (Krause et al., 1982), zeaxanthin-dependent quenching qZ (Dall'Osto et al., 2005, Nilkens et al., 2010) and photoinhibitory quenching, qI (Krause 1988) have been shown to contribute to NPQ; whereas quenching due to state transitions, qT, is considered to be a minor contributor to NPQ in saturating light (Nilkens et al., 2010). The relative contribution of each of these components in protecting PSII from photodamage and their occurrence under different conditions is not fully understood (Lambrev et al., 2012, Ruban 2016).

qE, also referred to as the flexible mode of energy dissipation, has been most extensively studied, and its key molecular players have been identified. In vascular plants, the protein PsbS senses acidification of the lumen upon light exposure and, together with the xanthophyll pigment zeaxanthin, is necessary to catalyze formation of a quenching site (Demmig et al., 1987, Niyogi et al., 1997, Li et al., 2000, Johnson and Ruban 2011, Sylak-Glassman et al., 2014). Previously, we have asked the question whether NPQ could be rescued in the absence of either of these key players. From a suppressor screen using the npq1 mutant lacking zeaxanthin, we found that the xanthophyll pigment lutein can partially replace the function of zeaxanthin (Li et al., 2009a). Through a suppressor screen using the npq4 mutant lacking PsbS, we uncovered a slowly inducible and reversible NPQ mechanism regulated by the suppressor of quenching 1 (SOQ1) protein (Brooks et al., 2013).

The soq1 mutant exhibits additional quenching compared to wild type. As the name indicates, the function of SOQ1 is to suppress the additional quenching that is otherwise observed when this protein is not functioning. This additional quenching is independent of known components required for NPQ such as PsbS, zeaxanthin, ΔpH formation or the STN7 kinase (Brooks et al., 2013) and is induced by light intensities greater than 1200 μmol photons m⁻² s⁻¹, so it is an example of a sustained pH-independent type of NPQ (Demmig-Adams et al., 2014). The maximum fluorescence, Fₘ, and the initial fluorescence, Fₒ, are both quenched by this mechanism suggesting its occurrence in the proximal or peripheral antenna of photosystem II (PSII). The peripheral antenna of PSII is composed of the light-harvesting, chlorophyll-binding Lhcb proteins, also referred to as LHCII, divided into minor (Lhcb4, 5, 6 or CP29, 26, 24, respectively) and major complexes (Lhcb1, 2, 3). These proteins are organized mostly into oligomers, such as the trimeric form for the major LHCII, or monomeric form for the minor LHCII proteins that link trimeric LHCII proteins to dimeric PSII core complexes forming PSII-LHCII supercomplexes (Ballottari et al., 2012).

SOQ1 is a chloroplast-localized membrane protein of 104 kDa that contains multiple domains including a HAD phosphatase on the stromal side of the thylakoid membrane, a transmembrane helix, and thioredoxin-like and β-propeller NHL domains on the lumenal side of the thylakoid membrane. The stromal domain is dispensable for SOQ1 to suppress this additional quenching, whereas the lumenal domains are required (Brooks et al., 2013).

### SUMMARY OF THE INVENTION

Nonphotochemical quenching (NPQ) comprises mechanisms by which photosynthetic organisms harmlessly dissipate excess absorbed light energy. Photoinhibitory quenching (qI), thought to be the result of photoinactivation of PSII, is the slowest component of NPQ to reverse and is the least understood. The possibility that part of qI may be photoprotective has been little examined, in part because of the lack of mutants directly affecting qI. In the model plant Arabidopsis thaliana, the soq1 mutant displays additional slowly reversible NPQ relative to wild type. To identify molecular players of this NPQ pathway, we screened for suppressors of soq1 that showed a low level of NPQ, and mutants affecting either chlorophyllide a oxygenase (CAO) or the chloroplastic lipocalin protein (CHL) were isolated. Mutants affecting CAO are devoid of oligomerized PSII peripheral antenna proteins (LHCII), strongly suggesting that the additional quenching observed in soq1 occurs in LHCII. We found that the CHL-dependent NPQ mechanism operates under stress conditions such as cold and high light, and our results suggest that SOQ1 inhibits CHL-dependent quenching under non-stress conditions. We propose that, under stress conditions, CHL protects the thylakoid membrane by forming quenching sites in the antenna of PSII, thereby preventing singlet oxygen stress.

Thus, in one embodiment, eliminating a sustained photoprotective mechanism (by mutating or silencing the CHL gene) may be used to improve photosynthesis of plants, thereby improving food or energy crop yield. Photoprotection competes with light harvesting, so by eliminating unnecessary light energy dissipation, more energy will be available for plant growth.

Crop yield improvement is predicted using this strategy that reroutes light energy to biomass instead of being dissipated. The involvement of the CHL gene in light energy dissipation was not known. Understanding the CHL gene and the molecular players involved in light energy dissipation provides for methods and compositions to improved photosynthesis. Furthermore, environmental conditions such as cold and high light provides key conditions under which the CHL-dependent photoprotective mechanism operates.

Therefore, in another embodiment, a polynucleotide encoding a mutant CHL protein, an expression cassette that incorporates the mutant CHL protein and/or a cell comprising this expression cassette in its genome. In another embodiment, a plant incorporating the cell comprising the expression cassette having the polynucleotide encoding the mutant CHL protein.

The present invention provides for a method for improving photosynthesis in a plant cell or plant, comprising the reducing the expression of a Chloroplastic Lipocalin (CHL) gene in a plant cell or plant whereby the plant cell or plant, when cultured or grown under conditions suitable for photosynthesis, increases photosynthesis within the plant cell or plant.

In some embodiments, the reducing step comprises mutating the CHL gene in the plant cell or plant such that the mutated CHL gene has reduced or no biological activity, reduced transcription of the CHL gene, or the CHL gene is knocked-out, or silencing the expression of the CHL gene through an introduced iRNA or antisense RNA construct in the plant cell or plant that is specific for the CHL gene.

The present invention provides for a method for improving photosynthesis in a plant, comprising the steps of eliminating a sustained photoprotective mechanism in a plant by mutating or silencing the Chloroplastic Lipocalin (CHL) gene whereby photosynthesis of the plants increases.

The present invention provides for a polynucleotide encoding a mutant CHL protein, wherein the mutant CHL protein has reduced or no biological activity.

In some embodiments, the mutant CHL protein is chl-2 (AtCHL-A255V).

In some embodiments, an open reading frame (ORF) encoding the mutant CHL protein is operatively linked to a promoter capable of transcribing the ORF encoding the mutant CHL protein.

The present invention provides for an expression cassette that incorporates the polynucleotide of the present invention and expresses a mutant CHL protein that has reduced or no biological activity.

The present invention provides for a cell comprising the expression cassette of the present invention in its genome.

The present invention provides for any of the novel compositions or methods taught hererin.

### BRIEF DESCRIPTION OF THE DRAWINGS

FIG. 1A. Strict suppressors of soq1 npq4 show similar level of NPQ as npq4. Plants were grown at 120 μmol photons m⁻² s⁻¹, induction of NPQ at 1200 μmol photons m⁻² s⁻¹(white bar) and relaxation in the dark (black bar). A) NPQ kinetics of npq4, soq1 npq4, strict suppressors A26 and A42 (M2 individuals).

FIG. 1B. Strict suppressors of soq1 npq4 show similar level of NPQ as npq4. Plants were grown at 120 μmol photons m⁻² s⁻¹, induction of NPQ at 1200 μmol photons m⁻² s⁻¹ (white bar) and relaxation in the dark (black bar). HPLC traces showing lack of chlorophyll b in mutants A26 and A42 (black arrow) after 1 h high light treatment (1000 μmol photons m⁻² s⁻¹) of detached leaves (pigments were extracted from same leaf area).

FIG. 1C. Strict suppressors of soq1 npq4 show similar level of NPQ as npq4. Plants were grown at 120 μmol photons m⁻² s⁻¹, induction of NPQ at 1200 μmol photons m⁻² s⁻¹ (white bar) and relaxation in the dark (black bar). NPQ kinetics of soq1 npq4, strict suppressors A205 and A252 and soq1 npq4× A205 F1. Data represent means+/−SD (n=3 F1 individuals). For A205 and A252: n=2 M3 individuals.

FIG. 2. Schematic representation of CHL protein with positions of mutations. Predicted chloroplast transit peptide (cTP) and lumen transit peptide (1TP) based on software prediction for cTP and mass spectrometry data (from PPDB) for 1TP suggesting a mature size of 29 kDa; black squares, “structurally conserved regions” of the lipocalin fold; diamonds, conserved cysteines; adapted from (Charron et al., 2005). Positions of three mutant alleles are depicted: T-DNA knock-out (KO) mutant (chl-1) described in (Levesque-Tremblay et al. 2009), CHL-A1a255Val (chl-2) and splice site (chl-3) mutants respectively from suppressor mutants A205 and A252 isolated in this study.

FIG. 3. soq1 chlKO exhibits wild-type NPQ. NPQ kinetics of Col-0, soq1, chlKO, soq1 chlKO and soq1/soq1 chlKO/CHL. The soq1 chlKO mutant was identified among the F2 progeny of the cross soq1-1×chlKO (T-DNA knock-out mutant). Data represent means+/−SD (n=7 F3 individuals soq1 chlKO and n=4 F2 individuals soq1/soq1 chlKO/CHL). Growth at 120 μmol photons m⁻² s⁻¹, induction of NPQ at 1200 μmol photons m⁻² s⁻¹ (white bar) and relaxation in the dark (black bar).

FIG. 4A. CHL protein accumulation in soq1 and suppressor mutants. Proteins were separated by SDS-PAGE and analyzed by immunodetection with antibodies against SOQ1 or CHL. Coomassie blue is shown as loading control. Isolated thylakoids+/−200 mM DTT from plants grown under standard conditions.

FIG. 4B. CHL protein accumulation in soq1 and suppressor mutants. Proteins were separated by SDS-PAGE and analyzed by immunodetection with antibodies against SOQ1 or CHL. Coomassie blue is shown as loading control. Whole cell extracts from plants kept in the dark for 14 h (+200 mM DTT).

FIG. 5A. CHL-dependent quenching occurs in cold and high light conditions. Maximum fluorescence (Fₘ) values. Fluorescence amplitude was measured with a PAM fluorometer on leaf discs of same area from plants grown at 120 μmol photons m⁻² s⁻¹, 21° C. (light grey bar) and after a cold and high light treatment for 8 h at 1070 μmol photons m⁻² s⁻¹, 12° C. (dark grey bar). Leaf discs were dark-acclimated for 40 min (on moist surface) to fully relax qE and part of qZ. Data represent means+/−SD (n=4 individuals of each genotype). Asterisk(s) marks significance level of *P<0.05, **P<0.01 or ****P<0.0001 relative to Col-0 by one-way ANOVA followed by Tukey's post hoc test performed on data set from cold and high light treatment (full results of Tukey's multiple comparisons test are shown in Tables 3 and 4).

FIG. 5B. CHL-dependent quenching occurs in cold and high light conditions. Initial fluorescence (F₀) values. Fluorescence amplitude was measured with a PAM fluorometer on leaf discs of same area from plants grown at 120 μmol photons m⁻² s⁻¹, 21° C. (light grey bar) and after a cold and high light treatment for 8 h at 1070 μmol photons m⁻² s⁻¹, 12° C. (dark grey bar). Leaf discs were dark-acclimated for 40 min (on moist surface) to fully relax qE and part of qZ. Data represent means+/−SD (n=4 individuals of each genotype). Asterisk(s) marks significance level of *P<0.05, **P<0.01 or ****P<0.0001 relative to Col-0 by one-way ANOVA followed by Tukey's post hoc test performed on data set from cold and high light treatment (full results of Tukey's multiple comparisons test are shown in Tables 3 and 4).

FIG. 6. Model for CHL-dependent quenching regulated by SOQ1. We propose that SOQ1 inhibits, directly or indirectly, CHL activity under non-stress conditions. When CHL is active, quenching sites indicated by red stars are produced in the peripheral antenna of PSII (updated from Brooks et al. (2013)). There is direct involvement of CHL in forming quenching sites. The quenching sites require lipid-OH.

FIG. 7. Confirmation that quenching occurs in PSII peripheral antenna. NPQ kinetics of chlorina1-3 and soq1 chlorina1-3. The soq1 chlorina1 mutant was identified among the F2 progeny of the cross soq1-1×chlorina1-3. Data represent means+/−SD (n=4). The standard conditions are growth at 120 μmol photons m-2 s-1 and induction of NPQ at 1200 μmol photons m-2 s-1, here growth at 20 μmol photons m-2 s-1 and induction of NPQ at 2000 μmol photons m-2 s-1 (white bar) and relaxation in the dark (black bar).

FIG. 8. A205 and A252 are allelic mutants. NPQ kinetics of npq4, soq1npq4, A205, A252 and A205×A252 F1. Data represent means+/−SD (n=3 F1 individuals from A205×A252 cross). For A205 and A252: n=2 M3 individuals. Growth at 120 μmol photons m-2 s-1 and induction of NPQ at 1200 μmol photons m-2 s-1 (white bar) and relaxation in the dark (black bar).

FIG. 9A. Chromosome 1. Detected single nucleotide polymorphisms (SNPs) from the pooled mutant F2 individuals, with same NPQ phenotype as A205, from soq1 npq4×A205 cross were filtered for quality and to remove SNPs present in the parental line (soq1 npq4). The remaining 267 SNPs were plotted with the allele frequency on the Y axis and position on the X axis for each chromosome. A region of enrichment was also observed at the beginning of chromosome 1 but this linkage was not confirmed since A252 did not share any genes containing SNPs with A205 in that region and may be an artifact resulting from the parental line containing segregating mutations from the previous round of EMS mutagenesis.

FIG. 9B. Chromosome 2. Detected single nucleotide polymorphisms (SNPs) from the pooled mutant F2 individuals, with same NPQ phenotype as A205, from soq1 npq4×A205 cross were filtered for quality and to remove SNPs present in the parental line (soq1 npq4). The remaining 267 SNPs were plotted with the allele frequency on the Y axis and position on the X axis for each chromosome.

FIG. 9C. Chromosome 3. The mutation in A205 is on chromosome 3. Detected single nucleotide polymorphisms (SNPs) from the pooled mutant F2 individuals, with same NPQ phenotype as A205, from soq1 npq4×A205 cross were filtered for quality and to remove SNPs present in the parental line (soq1 npq4). The remaining 267 SNPs were plotted with the allele frequency on the Y axis and position on the X axis for each chromosome. A region enriched for SNPs showing tight linkage to the mutant phenotype was identified on chromosome 3.

FIG. 9D. Chromosome 4. Detected single nucleotide polymorphisms (SNPs) from the pooled mutant F2 individuals, with same NPQ phenotype as A205, from soq1 npq4×A205 cross were filtered for quality and to remove SNPs present in the parental line (soq1 npq4). The remaining 267 SNPs were plotted with the allele frequency on the Y axis and position on the X axis for each chromosome.

FIG. 9E. Chromosome 5. Detected single nucleotide polymorphisms (SNPs) from the pooled mutant F2 individuals, with same NPQ phenotype as A205, from soq1 npq4×A205 cross were filtered for quality and to remove SNPs present in the parental line (soq1 npq4). The remaining 267 SNPs were plotted with the allele frequency on the Y axis and position on the X axis for each chromosome.

FIG. 10. Level of PsbO in soq1 is not decreased after cold and high light treatment. Proteins from whole cell extracts were separated by SDS/PAGE and analyzed by immunodetection with antibodies against SOQ1 and PsbO with the ATP synthase β-subunit as a loading control. Leaf discs of same area were taken from plants (same set of plants used in FIGS. 5A and 5B) grown at 120 μmol photons m-2 s-1, 21° C. (Col-0, soq1, chlKO and soq1 chlKO) and after a cold and high light treatment at 1070 μmol photons m-2 s-1, 12° C. for 8 h (T1).

FIG. 11A. Xanthophyll pigment content during cold high light treatment experiment. Neo, viola, anthera and zeaxanthin. Pigment content was measured from the leaf discs of same area used for fluorescence measurements in FIGS. 5A and 5B, from plants grown at 120 μmol photons m-2 s-1, 21° C. (Col-0, soq1, chlKO and soq1 chlKO) and after a cold and high light treatment for 8 h at 1070 μmol photons m-2 s-1, 12° C. (T1). Leaf discs were dark-acclimated for 40 min (on moist surface) to fully relax qE and part of qZ. As seen here, some zeaxanthin remains after this dark-acclimation treatment. Data represent means+/−SD (n=4).

FIG. 11B. Xanthophyll pigment content during cold high light treatment experiment. Lutein. Pigment content was measured from the leaf discs of same area used for fluorescence measurements in FIGS. 5A and 5B, from plants grown at 120 μmol photons m-2 s-1, 21° C. (Col-0, soq1, chlKO and soq1 chlKO) and after a cold and high light treatment for 8 h at 1070 μmol photons m-2 s-1, 12° C. (T1). Leaf discs were dark-acclimated for 40 min (on moist surface) to fully relax qE and part of qZ. As seen here, some zeaxanthin remains after this dark-acclimation treatment. Data represent means+/−SD (n=4).

FIG. 12. CHL-dependent quenching occurs in wild-type in cold and high light.

FIG. 13. Comparison of Col-0, soq1, chlKO, and soq1 chlKO mutants shows quenching is photoprotective. Of the strains tested, the soq1 mutant survives best.

FIG. 14. Total chlorophyll in Col-0, soq1, chlKO, and soq1 chlKO mutants.

FIG. 15. Protein accumulation in OTK1 overexpressors and controls.

FIG. 16A. Overexpression of OTK1 prevents CHL-dependent quenching from occurring. Protein accumulation in OTK1 overexpressors and controls.

FIG. 16B. Overexpression of OTK1 prevents CHL-dependent quenching from occurring. Growth of OTK1 overexpressors and controls at 7 weeks, grown under 120 μmol m-2 s-1, 10 h day/14 h night cycle.

FIG. 16C. Overexpression of OTK1 prevents CHL-dependent quenching from occurring. NPQ of OTK1 overexpressor and controls. Plants were dark-adapted for 20 min, induction of NPQ at 1200 μmol photons m-2 s-1 (white bar) and relaxation in the dark (black bar).

FIG. 17A. Overexpression of OTK1 prevents CHL-dependent quenching from occurring in soq1 otk1-3. Protein accumulation in OTK1 overexpressors and controls.

FIG. 17B. Overexpression of OTK1 prevents CHL-dependent quenching from occurring in soq1 otk1-3. Growth of OTK1 overexpressors and controls at 4.5 weeks, grown under 120 μmol m-2 s-1, 10 h day/14 h night cycle.

FIG. 17C. Overexpression of OTK1 prevents CHL-dependent quenching from occurring in soq1 otk1-3. NPQ of OTK1 overexpressors and controls. Plants were dark-adapted for 20 min, induction of NPQ at 1200 μmol photons m-2 s-1 (white bar) and relaxation in the dark (black bar).

### DETAILED DESCRIPTION OF THE INVENTION

To elucidate the mechanism of this newly described NPQ and identify possible targets of SOQ1, we performed a suppressor screen on the soq1 npq4 mutant and searched for mutants that no longer exhibited this slowly inducible and reversible NPQ. We proposed that SOQ1 is involved in reducing lumenal or lumen-exposed target proteins to prevent formation of a slowly reversible antenna quenching, either directly or via another protein (Brooks et al., 2013). We expect that suppressors (in the classical genetic definition) of the higher quenching observed in the soq1 npq4 mutant background might be mutated in the site of quenching (proximal or peripheral antenna potentially) or in a putative downstream target of SOQ1 (protein X depicted in the model of Brooks et al. (2013)). By definition, the NPQ phenotype of these suppressors (triple mutants) will return to the initial low NPQ phenotype, which is that of npq4. Two types of mutants emerged from the screen, one type affecting the peripheral antenna of PSII and one type identifying the likely downstream target of SOQ1. These findings confirm that this NPQ mechanism occurs in the antenna, specifically in the peripheral antenna of PSII, and uncover a role for the chloroplastic lipocalin in this quenching mechanism.

### Description of Sequences

Accession Numbers.

Sequence data from this article can be found in the Arabidopsis Genome Initiative under accession numbers At1g56500 (SOQ1), At3g47860 (CHL), At1g44575 (PsbS), At1g44446 (CAO), all of which are hereby incorporated by reference.

The CHL gene encodes chloroplastic lipocalin (AT3G47860) of Arabidopsis thaliana (ecotype: Columbia), having the lineage: Eukaryota; Viridiplantae; Streptophyta; Embryophyta; Tracheophyta; Spermatophyta; Magnoliophyta; eudicotyledons; Gunneridae; Pentapetalae; rosids; malvids; Brassicales; Brassicaceae; Camelineae; Arabidopsis; which is involved in the protection of thylakoidal membrane lipids against reactive oxygen species, especially singlet oxygen, produced upon excess light.

The genomic sequence of CHL chloroplastic lipocalin [Arabidopsis thaliana (thale cress)] (AT3G47860) has the following nucleotide sequence:

(SEQ ID NO: 1)
ATAAAAGTCTGTGGCGACCAAAAGAAGTGAGAGAGAAAGAGAGAGATATG
ATATTATTAAGTAGTAGTATAAGCCTCTCAAGACCAGTTTCTTCTCAAAG
CTTCTCTCCACCTGCTGCCACTTCAACAAGGTATGGTTTTATGTTTTGAA
TCACAACTTCATTAGCTCCGATTAACCTCAAGATCATTAATTTAGTTATA
GGTTTAGGAACAAAGTTACAGAATCAGATCCTGGAATGGAACTATCTTGG
ATTATAGAGATTCTTGGTTTACTGATCTCAGAAATTCTGTTTTCTCTTGG
TTTCACTTATGACAGGAGATCTCATTCCTCTGTCACAGTCAAGTGCTGCT
GTTCTTCCAGAAGGTTGTTGAAGAATCCTGAGTTAAAATGTTCCTTGGAG
AATCTCTTTGAAATCCAGGCTTTGAGGAAGTGTTTTGTTTCAGGGTTTGC
AGCTATTTTGCTTCTCTCTCAGGCAGGCCAGGTATGTTGGAGCCGACATT
GCTATAAGCATGTTCGATTGCAACCACAAGCGGTCTGAAATTTCTGAGTT
TCTTCTGGCAGGGTATAGCGTTGGATCTCTCATCTGGTTATCAGAACATT
TGCCAACTAGGGAGTGCTGCTGCTGTGGGAGAAAACAAGCTGACTCTTCC
ATCTGATGGTGACTCGGAATCAATGATGATGATGATGATGAGAGGCATGA
CTGCTAAGAACTTTGACCCTGTTAGGTACTCTGGAAGATGGTTTGAAGTA
GCTTCTCTTAAGCGTGGATTTGCAGGTCAAGGCCAAGAAGACTGTCATTG
CACTCAGGTAATTGTTTCTTCAAGTCCACAAATTGTGAAAACACATCCTC
TGCTAACATAGTGTGAGAACCTTTCAACTGTTGCAGGGAGTATACACGTT
TGATATGAAGGAATCAGCCATTAGAGTAGATACATTTTGTGTTCATGGCA
GCCCTGATGGATATATAACAGGAATCAGAGGGAAAGTTCAATGCGTGGGA
GCGGAAGACCTCGAGAAAAGCGAGACTGACTTAGAAAAGCAAGAGATGAT
TAAAGAGAAGTGTTTCCTACGATTTCCCACCATTCCTTTTATCCCCAAGT
TGCCTTATGATGTCATAGCCACAGACTACGACAACTACGCACTTGTTTCT
GGAGCCAAAGACAAGGGCTTTGTTCAGGTGATTCTAAGAGAAAGAACTCA
TGAGTTTGAGAAATATCTCTTGCACTGATCTCTTATATAAACCTAATGTG
TTTGATATGTTAGGTATACTCAAGGACGCCAAATCCAGGACCTGAGTTCA
TCGCAAAGTACAAGAACTACTTGGCACAATTTGGCTATGACCCGGAAAAA
ATAAAGGATACACCACAGGACTGTGAAGTGACTGATGCTGAGCTAGCAGC
CATGATGTCCATGCCAGGTATGGAGCAAACACTGACCAACCAGTTTCCAG
ATCTTGGATTAAGAAAGTCAGTCCAGTTTGATCCCTTCACAAGTGTGTTT
GAAACCTTGAAGAAACTTGTACCGCTCTATTTCAAATAGAGCAAGCTTCT
TTGCTCAAATTCTTATGTAGACTATAATCACTGTCCATATATACATATCT
TCCAGAATCAAAACACTCTTCTGATCATAACACCATTGATTAGAGAA

CHL chloroplastic lipocalin [Arabidopsis thaliana (thale cress)] (AT3G47860.1) has the following amino acid sequence:

(SEQ ID NO: 2)
MILLSSSISLSRPVSSQSFSPPAATSTRRSHSSVTVKCCCSSRRLLKNPE
LKCSLENLFEIQALRKCFVSGFAAILLLSQAGQGIALDLSSGYQNICQLG
SAAAVGENKLTLPSDGDSESMMMMMMRGMTAKNFDPVRYSGRWFEVASLK
RGFAGQGQEDCHCTQGVYTFDMKESAIRVDTFCVHGSPDGYITGIRGKVQ
CVGAEDLEKSETDLEKQEMIKEKCFLRFPTIPFIPKLPYDVIATDYDNYA
LVSGAKDKGFVQVYSRTPNPGPEFIAKYKNYLAQFGYDPEKIKDTPQDCE
VTDAELAAMMSMPGMEQTLTNQFPDLGLRKSVQFDPFTSVFETLKKLVPL
YFK*

OKT1 (At4g31530) has the following nucleotide sequence:

(SEQ ID NO: 3)
AAATTAAATAATGGCTTCTCCTTAAAATTATCTAAGCTCGCTTGACCAAT
CATGGCTACCACCACCAATCTCAGCTTCGCTCCTCCCTCTTACTCCCGTT
TTGCCGCTACAAAATCCCAAATCAGAAACCCTCTGTTTACGTCCCCCCTG
CCACTCCCATCTTCTTTCTTCCTGGTTAGAAATGAAGCATCTTTATCTTC
GTCAATCACGCCAGTCCAAGCTTTTACAGAAGAAGCCGTCGATACTTCGG
ATTTAGCTTCTTCTTCTTCAAAGCTTGTACTTGTCGTTGGCGGCACTGGT
GGTGTAGGTACTTAAATGTTCAATTTTGAAATTAGGACTATGAATTTCTC
ACTATTGGGTCTGCTGAATGATGGTTGTTTTAAAGTCTTATCCTTTTTGG
TATCAGGTCAACTTGTGGTAGCTTCATTGCTCAAGAGGAATATAAGATCA
AGGTTATTACTGCGTGATCTTGACAAAGCTACCAAGTTATTTGGCAAACA
AGATGAATATTCCTTGCAGGTTCGGACTTAATCTCACTATTTCGGATACA
CTACTTGTTCTTTTTATACGATAAGTTTAAACCATGTTGTTATAGGTAGT
TAAAGGGGATACTAGGAATGCAGAGGATCTTGATCCATCCATGTTTGAGG
TTCGTCTTCTTGCTCCTGTTTTGTTTGGCACGAGTTTCACTTGTTCTTCA
TTTGATTTTTAACTGATTTGCCTAGGGTGTCACACATGTGATTTGTACCA
CTGGAACTACAGCTTTTCCTTCTAAGAGGTGGAATGAAGAAAACACTCCT
GAGAAAGTAGGTGAGAACACATTTCTTTCCAATTAAGAACGTTAGATGTC
TTGTTTTGTTATCAGTTCTTTCTTACATGTCATGATCGATTTTCCTTTTT
GGGACTATGTGTTTTTTTTTGTGTTCAAAAAGATTGGGAAGGTGTGAAGA
ATCTCATTTCAGCATTGCCATCATCGGTGAAGAGAGTTGTTTTGGTTTCA
TCAGTAGGTGTGACCAAGTCTAATGAGCTACCCTGGAGGTGAGACTAGAG
TATCTCCGCTAATTTACTTTAGTCTGATTGTCTCTACGTGTAGTTTATTT
GCTGAATGTATGTTTTGTTTGACAGCATCATGAACCTTTTTGGAGTTCTT
AAGTACAAGAAGATGGGGGAAGATTTTCTTCGTGACTCTGGTCTTCCATT
CACCATTATCAGGTTTCGAACAAAAGAGTTACCCACTTTTTTTGCTCTTA
ATACTCCAAAACGCAGGCAACATATGGTTTACTGATTTACTGTAATGTGA
TGTTTTCTTACCAGACCTGGTAGATTGACTGATGGACCATACACATCTTA
TGATCTGAATACTTTGCTCAAAGCTACAGCTGGTGAAAGGCGTGCAGTTG
TTATTGGTCAAGGTAAATTTGAGATTCTTTGATGGTCTTGCAGCACTAAG
ATTTTTGAGATTACTTTATTTACATAATGGCTCCTTTTAACAGGGGACAA
CCTTGTTGGAGAGGTAAGTAGACTTGTAGTGGCTGAAGCTTGTATACAGG
CACTTGATATTGAATTCACACAAGGCAAAGCTTACGAGATCAATTCAGTA
AAGGTACCACAAACAGTTTCCTTGAAATTTGAACAAAGTGGAAATGTAGT
CTGATCAAGAAACAATGTCTTAAGGGGTGTCCTGGAGATTCAAAGCTTTA
GTTGATCATTGAAAATGTTTCAAAGATTTGGTTGCCATTTTTTTATATGT
TTGAGAAATGTGGTTTCAGGGGGATGGTCCAGGAAGTGATCCACAGCAAT
GGCGAGAGTTGTTTAAAGCTGCAGAATCCAAATGACAAAAGAGGACTTTT
GAGAGATGTGTACAGAATTGTTAGCGAGACATTACATATATGGTCGATTG
TGTATACATGTGCTTTCTTTTGGTCTTTGACTTCATCATTACTGTAATTA
CTTTATCTATAACTAGAAGTTCTTTCTTGCAAATCAA

OKT1 has the following amino acid sequence:

(SEQ ID NO: 4)
MATTTNLSFAPPSYSRFAATKSQIRNPLFTSPLPLPSSFFLVRNEASLSS
SITPVQAFTEEAVDTSDLASSSSKLVLVVGGTGGVGQLVVASLLKRNIRS
RLLLRDLDKATKLFGKQDEYSLQVVKGDTRNAEDLDPSMFEGVTHVICTT
GTTAFPSKRWNEENTPEKVDWEGVKNLISALPSSVKRVVLVSSVGVTKSN
ELPWSIMNLFGVLKYKKMGEDFLRDSGLPFTIIRPGRLTDGPYTSYDLNT
LLKATAGERRAVVIGQGDNLVGEVSRLVVAEACIQALDIEFTQGKAYEIN
SVKVPQTVSLKFEQSGNGDGPGSDPQQWRELFKAAESK

The nucleotide sequence of the pEarleyGate 100 expression vector comprising a 35S promoter (underlined)-OTK1cDNA (bold)-Flag tag (italics) is as follows:

(SEQ ID NO: 5)
TGGCAGGATATATTGTGGTGTAAACAAATTGACGCTTAGACAACTTAATAACACATTGCGGACG
TTTTTAATGTACTGAATTAACGCCGAATTAATTCGAGCTCGGATCTGATAATTTATTTGAAAAT
TCATAAGAAAAGCAAACGTTACATGAATTGATGAAACAATACAAAGACAGATAAAGCCACGCAC
ATTTAGGATATTGGCCGAGATTACTGAATATTGAGTAAGATCACGGAATTTCTGACAGGAGCAT
GTCTTCAATTCAGCCCAAATGGCAGTTGAAATACTCAAACCGCCCCATATGCAGGAGCGGATCA
TTCATTGTTTGTTTGGTTGCCTTTGCCAACATGGGAGTCCAAGATTCTGCAGTCAAATCTCGGT
GACGGGCAGGACCGGACGGGGCGGTACCGGCAGGCTGAAGTCCAGCTGCCAGAAACCCACGTCA
TGCCAGTTCCCGTGCTTGAAGCCGGCCGCCCGCAGCATGCCGCGGGGGGCATATCCGAGCGCCT
CGTGCATGCGCACGCTCGGGTCGTTGGGCAGCCCGATGACAGCGACCACGCTCTTGAAGCCCTG
TGCCTCCAGGGACTTCAGCAGGTGGGTGTAGAGCGTGGAGCCCAGTCCCGTCCGCTGGTGGCGG
GGGGAGACGTACACGGTCGACTCGGCCGTCCAGTCGTAGGCGTTGCGTGCCTTCCAGGGGCCCG
CGTAGGCGATGCCGGCGACCTCGCCGTCCACCTCGGCGACGAGCCAGGGATAGCGCTCCCGCAG
ACGGACGAGGTCGTCCGTCCACTCCTGCGGTTCCTGCGGCTCGGTACGGAAGTTGACCGTGCTT
GTCTCGATGTAGTGGTTGACGATGGTGCAGACCGCCGGCATGTCCGCCTCGGTGGCACGGCGGA
TGTCGGCCGGGCGTCGTTCTGGGCTCATCGATTCGATTTGGTGTATCGAGATTGGTTATGAAAT
TCAGATGCTAGTGTAATGTATTGGTAATTTGGGAAGATATAATAGGAAGCAAGGCTATTTATCC
ATTTCTGAAAAGGCGAAATGGCGTCACCGCGAGCGTCACGCGCATTCCGTTCTTGCTGTAAAGC
GTTGTTTGGTACACTTTTGACTAGCGAGGCTTGGCGTGTCAGCGTATCTATTCAAAAGTCGTTA
ATGGCTGCGGATCAAGAAAAAGTTGGAATAGAAACAGAATACCCGCGAAATTCAGGCCCGGTTG
CCATGTCCTACACGCCGAAATAAACGACCAAATTAGTAGAAAAATAAAAACTGACTCGGATACT
TACGTCACGTCTTGCGCACTGATTTGAAAAATCTCAGAATTCCAATCCCACAAAAATCTGAGCT
TAACAGCACAGTTGCTCCTCTCAGAGCAGAATCGGGTATTCAACACCCTCATATCAACTACTAC
GTTGTGTATAACGGTCCACATGCCGGTATATACGATGACTGGGGTTGTACAAAGGCGGCAACAA
ACGGCGTTCCCGGAGTTGCACACAAGAAATTTGCCACTATTACAGAGGCAAGAGCAGCAGCTGA
CGCGTACACAACAAGTCAGCAAACAGACAGGTTGAACTTCATCCCCAAAGGAGAAGCTCAACTC
AAGCCCAAGAGCTTTGCTAAGGCCCTAACAAGCCCACCAAAGCAAAAAGCCCACTGGCTCACGC
TAGGAACCAAAAGGCCCAGCAGTGATCCAGCCCCAAAAGAGATCTCCTTTGCCCCGGAGATTAC
AATGGACGATTTCCTCTATCTTTACGATCTAGGAAGGAAGTTCGAAGGTGAAGGTGACGACACT
ATGTTCACCACTGATAATGAGAAGGTTAGCCTCTTCAATTTCAGAAAGAATGCTGACCCACAGA
TGGTTAGAGAGGCCTACGCAGCAGGTCTCATCAAGACGATCTACCCGAGTAACAATCTCCAGGA
GATCAAATACCTTCCCAAGAAGGTTAAAGATGCAGTCAAAAGATTCAGGACTAATTGCATCAAG
AACACAGAGAAAGACATATTTCTCAAGATCAGAAGTACTATTCCAGTATGGACGATTCAAGGCT
TGCTTCATAAACCAAGGCAAGTAATAGAGATTGGAGTCTCTAAAAAGGTAGTTCCTACTGAATC
TAAGGCCATGCATGGAGTCTAAGATTCAAATCGAGGATCTAACAGAACTCGCCGTGAAGACTGG
CGAACAGTTCATACAGAGTCTTTTACGACTCAATGACAAGAAGAAAATCTTCGTCAACATGGTG
GAGCACGACACTCTGGTCTACTCCAAAAATGTCAAAGATACAGTCTCAGAAGACCAAAGGGCTA
T
TGAGACTTTTCAACAAAGGATAATTTCGGGAAACCTCCTCGGATTCCATTGCCCAGCTATCTGT
CACTTCATCGAAAGGACAGTAGAAAAGGAAGGTGGCTCCTACAAATGCCATCATTGCGATAAAG
GAAAGGCTATCATTCAAGATCTCTCTGCCGACAGTGGTCCCAAAGATGGACCCCCACCCACGAG
GAGCATCGTGGAAAAAGAAGACGTTCCAACCACGTCTTCAAAGCAAGTGGATTGATGTGACATC
TCCACTGACGTAAGGGATGACGCACAATCCCACTATCCTTCGCAAGACCCTTCCTCTATATAAG
GAAGTTCATTTCATTTGGAGAGGACACGCTCGAGATC
ATGGCTACCACCACCAATCTCAGCTTCGCTCCTCCCTCTTACTCCCGTTTTGCCGCTACAAAAT
CCCAAATCAGAAACCCTCTGTTTACGTCCCCCCTGCCACTCCCATCTTCTTTCTTCCTGGTTAG
AAATGAAGCATCTTTATCTTCGTCAATCACGCCAGTCCAAGCTTTTACAGAAGAAGCCGTCGAT
ACTTCGGATTTAGCTTCTTCTTCTTCAAAGCTTGTACTTGTCGTTGGCGGCACTGGTGGTGTAG
GTCAACTTGTGGTAGCTTCATTGCTCAAGAGGAATATAAGATCAAGGTTATTACTGCGTGATCT
TGACAAAGCTACCAAGTTATTTGGCAAACAAGATGAATATTCCTTGCAGGTAGTTAAAGGGGAT
ACTAGGAATGCAGAGGATCTTGATCCATCCATGTTTGAGGGTGTCACACATGTGATTTGTACCA
CTGGAACTACAGCTTTTCCTTCTAAGAGGTGGAATGAAGAAAACACTCCTGAGAAAGTAGATTG
GGAAGGTGTGAAGAATCTCATTTCAGCATTGCCATCATCGGTGAAGAGAGTTGTTTTGGTTTCA
TCAGTAGGTGTGACCAAGTCTAATGAGCTACCCTGGAGCATCATGAACCTTTTTGGAGTTCTTA
AGTACAAGAAGATGGGGGAAGATTTTCTTCGTGACTCTGGTCTTCCATTCACCATTATCAGACC
TGGTAGATTGACTGATGGACCATACACATCTTATGATCTGAATACTTTGCTCAAAGCTACAGCT
GGTGAAAGGCGTGCAGTTGTTATTGGTCAAGGGGACAACCTTGTTGGAGAGGTAAGTAGACTTG
TAGTGGCTGAAGCTTGTATACAGGCACTTGATATTGAATTCACACAAGGCAAAGCTTACGAGAT
CAATTCAGTAAAGGTACCACAAACAGTTTCCTTGAAATTTGAACAAAGTGGAAATGGGGATGGT
CCAGGAAGTGATCCACAGCAATGGCGAGAGTTGTTTAAAGCTGCAGAATCCAAATGA

[symbol]

GCCTAGGTGAGTCTAGAGAGTTAATTAAGACCCGGGACTAGTCCCTAGAGTCCTGCTTTAATGA
GATATGCGAGACGCCTATGATCGCATGATATTTGCTTTCAATTCTGTTGTGCACGTTGTAAAAA
ACCTGAGCATGTGTAGCTCAGATCCTTACCGCCGGTTTCGGTTCATTCTAATGAATATATCACC
CGTTACTATCGTATTTTTATGAATAATATTCTCCGTTCAATTTACTGATTGTACCCTACTACTT
ATATGTACAATATTAAAATGAAAACAATATATTGTGCTGAATAGGTTTATAGCGACATCTATGA
TAGAGCGCCACAATAACAAACAATTGCGTTTTATTATTACAAATCCAATTTTAAAAAAAGCGGC
AGAACCGGTCAAACCTAAAAGACTGATTACATAAATCTTATTCAAATTTCAAAAGTGCCCCAGG
GGCTAGTATCTACGACACACCGAGCGGCGAACTAATAACGCTCACTGAAGGGAACTCCGGTTCC
CCGCCGGCGCGCATGGGTGAGATTCCTTGAAGTTGAGTATTGGCCGTCCGCTCTACCGAAAGTT
ACGGGCACCATTCAACCCGGTCCAGCACGGCGGCCGGGTAACCGACTTGCTGCCCCGAGAATTA
TGCAGCATTTTTTTGGTGTATGTGGGCCCCAAATGAAGTGCAGGTCAAACCTTGACAGTGACGA
CAAATCGTTGGGCGGGTCCAGGGCGAATTTTGCGACAACATGTCGAGGCTCAGCAGGACCTGCA
GGCATGCAAGCTTGGCACTGGCCGTCGTTTTACAACGTCGTGACTGGGAAAACCCTGGCGTTAC
CCAACTTAATCGCCTTGCAGCACATCCCCCTTTCGCCAGCTGGCGTAATAGCGAAGAGGCCCGC
ACCGATCGCCCTTCCCAACAGTTGCGCAGCCTGAATGGCGAATGCTAGAGCAGCTTGAGCTTGG
ATCAGATTGTCGTTTCCCGCCTTCAGTTTAAACTATCAGTGTTTGACAGGATATATTGGCGGGT
AAACCTAAGAGAAAAGAGCGTTTATTAGAATAACGGATATTTAAAAGGGCGTGAAAAGGTTTAT
CCGTTCGTCCATTTGTATGTGCATGCCAACCACAGGGTTCCCCTCGGGATCAAAGTACTTTGAT
CCAACCCCTCCGCTGCTATAGTGCAGTCGGCTTCTGACGTTCAGTGCAGCCGTCTTCTGAAAAC
GACATGTCGCACAAGTCCTAAGTTACGCGACAGGCTGCCGCCCTGCCCTTTTCCTGGCGTTTTC
TTGTCGCGTGTTTTAGTCGCATAAAGTAGAATACTTGCGACTAGAACCGGAGACATTACGCCAT
GAACAAGAGCGCCGCCGCTGGCCTGCTGGGCTATGCCCGCGTCAGCACCGACGACCAGGACTTG
ACCAACCAACGGGCCGAACTGCACGCGGCCGGCTGCACCAAGCTGTTTTCCGAGAAGATCACCG
GCACCAGGCGCGACCGCCCGGAGCTGGCCAGGATGCTTGACCACCTACGCCCTGGCGACGTTGT
GACAGTGACCAGGCTAGACCGCCTGGCCCGCAGCACCCGCGACCTACTGGACATTGCCGAGCGC
ATCCAGGAGGCCGGCGCGGGCCTGCGTAGCCTGGCAGAGCCGTGGGCCGACACCACCACGCCGG
CCGGCCGCATGGTGTTGACCGTGTTCGCCGGCATTGCCGAGTTCGAGCGTTCCCTAATCATCGA
CCGCACCCGGAGCGGGCGCGAGGCCGCCAAGGCCCGAGGCGTGAAGTTTGGCCCCCGCCCTACC
CTCACCCCGGCACAGATCGCGCACGCCCGCGAGCTGATCGACCAGGAAGGCCGCACCGTGAAAG
AGGCGGCTGCACTGCTTGGCGTGCATCGCTCGACCCTGTACCGCGCACTTGAGCGCAGCGAGGA
AGTGACGCCCACCGAGGCCAGGCGGCGCGGTGCCTTCCGTGAGGACGCATTGACCGAGGCCGAC
GCCCTGGCGGCCGCCGAGAATGAACGCCAAGAGGAACAAGCATGAAACCGCACCAGGACGGCCA
GGACGAACCGTTTTTCATTACCGAAGAGATCGAGGCGGAGATGATCGCGGCCGGGTACGTGTTC
GAGCCGCCCGCGCACGTCTCAACCGTGCGGCTGCATGAAATCCTGGCCGGTTTGTCTGATGCCA
AGCTGGCGGCCTGGCCGGCCAGCTTGGCCGCTGAAGAAACCGAGCGCCGCCGTCTAAAAAGGTG
ATGTGTATTTGAGTAAAACAGCTTGCGTCATGCGGTCGCTGCGTATATGATGCGATGAGTAAAT
AAACAAATACGCAAGGGGAACGCATGAAGGTTATCGCTGTACTTAACCAGAAAGGCGGGTCAGG
CAAGACGACCATCGCAACCCATCTAGCCCGCGCCCTGCAACTCGCCGGGGCCGATGTTCTGTTA
GTCGATTCCGATCCCCAGGGCAGTGCCCGCGATTGGGCGGCCGTGCGGGAAGATCAACCGCTAA
CCGTTGTCGGCATCGACCGCCCGACGATTGACCGCGACGTGAAGGCCATCGGCCGGCGCGACTT
CGTAGTGATCGACGGAGCGCCCCAGGCGGCGGACTTGGCTGTGTCCGCGATCAAGGCAGCCGAC
TTCGTGCTGATTCCGGTGCAGCCAAGCCCTTACGACATATGGGCCACCGCCGACCTGGTGGAGC
TGGTTAAGCAGCGCATTGAGGTCACGGATGGAAGGCTACAAGCGGCCTTTGTCGTGTCGCGGGC
GATCAAAGGCACGCGCATCGGCGGTGAGGTTGCCGAGGCGCTGGCCGGGTACGAGCTGCCCATT
CTTGAGTCCCGTATCACGCAGCGCGTGAGCTACCCAGGCACTGCCGCCGCCGGCACAACCGTTC
TTGAATCAGAACCCGAGGGCGACGCTGCCCGCGAGGTCCAGGCGCTGGCCGCTGAAATTAAATC
AAAACTCATTTGAGTTAATGAGGTAAAGAGAAAATGAGCAAAAGCACAAACACGCTAAGTGCCG
GCCGTCCGAGCGCACGCAGCAGCAAGGCTGCAACGTTGGCCAGCCTGGCAGACACGCCAGCCAT
GAAGCGGGTCAACTTTCAGTTGCCGGCGGAGGATCACACCAAGCTGAAGATGTACGCGGTACGC
CAAGGCAAGACCATTACCGAGCTGCTATCTGAATACATCGCGCAGCTACCAGAGTAAATGAGCA
AATGAATAAATGAGTAGATGAATTTTAGCGGCTAAAGGAGGCGGCATGGAAAATCAAGAACAAC
CAGGCACCGACGCCGTGGAATGCCCCATGTGTGGAGGAACGGGCGGTTGGCCAGGCGTAAGCGG
CTGGGTTGTCTGCCGGCCCTGCAATGGCACTGGAACCCCCAAGCCCGAGGAATCGGCGTGAGCG
GTCGCAAACCATCCGGCCCGGTACAAATCGGCGCGGCGCTGGGTGATGACCTGGTGGAGAAGTT
GAAGGCCGCGCAGGCCGCCCAGCGGCAACGCATCGAGGCAGAAGCACGCCCCGGTGAATCGTGG
CAAGCGGCCGCTGATCGAATCCGCAAAGAATCCCGGCAACCGCCGGCAGCCGGTGCGCCGTCGA
TTAGGAAGCCGCCCAAGGGCGACGAGCAACCAGATTTTTTCGTTCCGATGCTCTATGACGTGGG
CACCCGCGATAGTCGCAGCATCATGGACGTGGCCGTTTTCCGTCTGTCGAAGCGTGACCGACGA
GCTGGCGAGGTGATCCGCTACGAGCTTCCAGACGGGCACGTAGAGGTTTCCGCAGGGCCGGCCG
GCATGGCCAGTGTGTGGGATTACGACCTGGTACTGATGGCGGTTTCCCATCTAACCGAATCCAT
GAACCGATACCGGGAAGGGAAGGGAGACAAGCCCGGCCGCGTGTTCCGTCCACACGTTGCGGAC
GTACTCAAGTTCTGCCGGCGAGCCGATGGCGGAAAGCAGAAAGACGACCTGGTAGAAACCTGCA
TTCGGTTAAACACCACGCACGTTGCCATGCAGCGTACGAAGAAGGCCAAGAACGGCCGCCTGGT
GACGGTATCCGAGGGTGAAGCCTTGATTAGCCGCTACAAGATCGTAAAGAGCGAAACCGGGCGG
CCGGAGTACATCGAGATCGAGCTAGCTGATTGGATGTACCGCGAGATCACAGAAGGCAAGAACC
CGGACGTGCTGACGGTTCACCCCGATTACTTTTTGATCGATCCCGGCATCGGCCGTTTTCTCTA
CCGCCTGGCACGCCGCGCCGCAGGCAAGGCAGAAGCCAGATGGTTGTTCAAGACGATCTACGAA
CGCAGTGGCAGCGCCGGAGAGTTCAAGAAGTTCTGTTTCACCGTGCGCAAGCTGATCGGGTCAA
ATGACCTGCCGGAGTACGATTTGAAGGAGGAGGCGGGGCAGGCTGGCCCGATCCTAGTCATGCG
CTACCGCAACCTGATCGAGGGCGAAGCATCCGCCGGTTCCTAATGTACGGAGCAGATGCTAGGG
CAAATTGCCCTAGCAGGGGAAAAAGGTCGAAAAGGTCTCTTTCCTGTGGATAGCACGTACATTG
GGAACCCAAAGCCGTACATTGGGAACCGGAACCCGTACATTGGGAACCCAAAGCCGTACATTGG
GAACCGGTCACACATGTAAGTGACTGATATAAAAGAGAAAAAAGGCGATTTTTCCGCCTAAAAC
TCTTTAAAACTTATTAAAACTCTTAAAACCCGCCTGGCCTGTGCATAACTGTCTGGCCAGCGCA
CAGCCGAAGAGCTGCAAAAAGCGCCTACCCTTCGGTCGCTGCGCTCCCTACGCCCCGCCGCTTC
GCGTCGGCCTATCGCGGCCGCTGGCCGCTCAAAAATGGCTGGCCTACGGCCAGGCAATCTACCA
GGGCGCGGACAAGCCGCGCCGTCGCCACTCGACCGCCGGCGCCCACATCAAGGCACCCTGCCTC
GCGCGTTTCGGTGATGACGGTGAAAACCTCTGACACATGCAGCTCCCGGAGACGGTCACAGCTT
GTCTGTAAGCGGATGCCGGGAGCAGACAAGCCCGTCAGGGCGCGTCAGCGGGTGTTGGCGGGTG
TCGGGGCGCAGCCATGACCCAGTCACGTAGCGATAGCGGAGTGTATACTGGCTTAACTATGCGG
CATCAGAGCAGATTGTACTGAGAGTGCACCATATGCGGTGTGAAATACCGCACAGATGCGTAAG
GAGAAAATACCGCATCAGGCGCTCTTCCGCTTCCTCGCTCACTGACTCGCTGCGCTCGGTCGTT
CGGCTGCGGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGGTCGTTCGCTCCAAG
CTGGGCTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGGATTAG
CAGAGCGAGGTATGTAGGCGGTGCTACAGAGTTCTTGAAGTGGTGGCCTAACTACGGCTACACT
AGAAGGACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAGAT
TACGCGCAGAAAAAAAGGATCTCAAGAAGATCCTTTGATCTTTTCTACGGGGTCTGACGCTCAG
TGGAACGAAAACTCACGTTAAGGGATTTTGGTCATGCATTCTAGGTACTAAAACAATTCATCCA
GTAAAATATAATATTTTATTTTCTCCCAATCAGGCTTGATCCCCAGTAAGTCAAAAAATAGCTC
GACATACTGTTCTTCCCCGATATCCTCCCTGATCGACCGGACGCAGAAGGCAATGTCATACCAC
TTGTCCGCCCTGCCGCTTCTCCCAAGATCAATAAAGCCACTTACTTTGCCATCTTTCACAAAGA
TGTTGCTGTCTCCCAGGTCGCCGTGGGAAAAGACAAGTTCCTCTTCGGGCTTTTCCGTCTTTAA
AAAATCATACAGCTCGCGCGGATCTTTAAATGGAGTGTCTTCTTCCCAGTTTTCGCAATCCACA
TCGGCCAGATCGTTATTCAGTAAGTAATCCAATTCGGCTAAGCGGCTGTCTAAGCTATTCGTAT
AGGGACAATCCGATATGTCGATGGAGTGAAAGAGCCTGATGCACTCCGCATACAGCTCGATAAT
CTTTTCAGGGCTTTGTTCATCTTCATACTCTTCCGAGCAAAGGACGCCATCGGCCTCACTCATG
AGCAGATTGCTCCAGCCATCATGCCGTTCAAAGTGCAGGACCTTTGGAACAGGCAGCTTTCCTT
CCAGCCATAGCATCATGTCCTTTTCCCGTTCCACATCATAGGTGGTCCCTTTATACCGGCTGTC
CGTCATTTTTAAATATAGGTTTTCATTTTCTCCCACCAGCTTATATACCTTAGCAGGAGACATT
CCTTCCGTATCTTTTACGCAGCGGTATTTTTCGATCAGTTTTTTCAATTCCGGTGATATTCTCA
TTTTAGCCATTTATTATTTCCTTCCTCTTTTCTACAGTATTTAAAGATACCCCAAGAAGCTAAT
TATAACAAGACGAACTCCAATTCACTGTTCCTTGCATTCTAAAACCTTAAATACCAGAAAACAG
CTTTTTCAAAGTTGTTTTCAAAGTTGGCGTATAACATAGTATCGACGGAGCCGATTTTGAAACC
GCGGTGATCACAGGCAGCAACGCTCTGTCATCGTTACAATCAACATGCTACCCTCCGCGAGATC
ATCCGTGTTTCAAACCCGGCAGCTTAGTTGCCGTTCTTCCGAATAGCATCGGTAACATGAGCAA
AGTCTGCCGCCTTACAACGGCTCTCCCGCTGACGCCGTCCCGGACTGATGGGCTGCCTGTATCG
AGTGGTGATTTTGTGCCGAGCTGCCGGTCGGGGAGCTGTTGGCTGGCTGG

### REFERENCES CITED

Adams III, W. W., Demmig-Adams, B., Rosenstiel, T. N., Brightwell, A. K. and Ebbert, V. (2002). Photosynthesis and Photoprotection in Overwintering Plants. Plant Biology 4: 545-557.
Aro, E. M., Virgin, I. and Andersson, B. (1993). Photoinhibition of Photosystem II. Inactivation, protein damage and turnover. Biochim Biophys Acta 1143: 113-134.
Ballottari, M., Girardon, J., Dall'Osto, L. and Bassi, R. (2012). Evolution and functional properties of Photosystem II light harvesting complexes in eukaryotes. Biochimica et Biophysica Acta (BBA)-Bioenergetics 1817: 143-157.
Boca, S., Koestler, F., Ksas, B., Chevalier, A., Leymarie, J., Fekete, A., Mueller, M. J. and Havaux, M. (2014). Arabidopsis lipocalins AtCHL and AtTIL have distinct but overlapping functions essential for lipid protection and seed longevity. Plant Cell Environ 37: 368-381.
Brooks, M. D., Sylak-Glassman, E. J., Fleming, G. R. and Niyogi, K. K. (2013). A thioredoxin-like/beta-propeller protein maintains the efficiency of light harvesting in Arabidopsis. Proc Natl Acad Sci USA 110: 2733-2740.
Bugos, R. C., Hieber, A. D. and Yamamoto, H. Y. (1998). Xanthophyll cycle enzymes are members of the lipocalin family, the first identified from plants. J Biol Chem 273: 15321-15324.
Casazza, A. P., Tarantino, D. and Soave, C. (2001). Preparation and functional characterization of thylakoids from Arabidopsis thaliana. Photosynth Res 68: 175-180.
Charron, J. B., Ouellet, F., Pelletier, M., Danyluk, J., Chauve, C. and Sarhan, F. (2005). Identification, expression, and evolutionary analyses of plant lipocalins. Plant Physiol 139: 2017-2028.
Dall'Osto, L., Caffarri, S. and Bassi, R. (2005). A mechanism of nonphotochemical energy dissipation, independent from PsbS, revealed by a conformational change in the antenna protein CP26. Plant Cell 17: 1217-1232.
Demmig, B. and Bjorkman, O. (1987). Comparison of the effect of excessive light on chlorophyll fluorescence (77K) and photon yield of O₂ evolution in leaves of higher plants. Planta 171: 171-184.
Demmig, B., Winter, K., Kruger, A. and Czygan, F. C. (1987). Photoinhibition and zeaxanthin formation in intact leaves: a possible role of the xanthophyll cycle in the dissipation of excess light energy. Plant Physiol 84: 218-224.
Demmig-Adams, B. and Adams, W. W., 3rd (2006). Photoprotection in an ecological context: the remarkable complexity of thermal energy dissipation. New Phytol 172: 11-21.
Demmig-Adams, B., Ebbert, V., Mellman, D. L., Mueh, K. E., Schaffer, L., Funk, C., Zarter, C. R., Adamska, I., Jansson, S. and Adams, W. W. (2006). Modulation of PsbS and flexible vs sustained energy dissipation by light environment in different species. Physiologia Plantarum 127: 670-680.
Demmig-Adams, B., Koh, S.-C., Cohu, C. M., Muller, O., Stewart, J. J. and Adams III, W. W. (2014). Non-Photochemical Fluorescence Quenching in Contrasting Plant Species and Environments. Non-Photochemical Quenching and Energy Dissipation in Plants, Algae and Cyanobacteria, Springer: 531-552.
Edelman, M. and Mattoo, A. K. (2008). D1-protein dynamics in photosystem II: the lingering enigma. Photosynthesis Research 98: 609-620.
Espineda, C. E., Linford, A. S., Devine, D. and Brusslan, J. A. (1999). The AtCAO gene, encoding chlorophyll a oxygenase, is required for chlorophyll b synthesis in Arabidopsis thaliana. Proc Natl Acad Sci USA 96: 10507-10511.
Flower, D. R. (1996). The lipocalin protein family: structure and function. Biochem J 318: 1-14.
Frenette Charron, J. B., Breton, G., Badawi, M. and Sarhan, F. (2002). Molecular and structural analyses of a novel temperature stress-induced lipocalin from wheat and Arabidopsis. FEBS Lett 517: 129-132.
Frenefe Charron J B, et al. (2005) Identification, expression, and evolutionary analyses of plant lipocalins. Plant Physiology 139, 2017-2028.
Gilmore, A. M. and Ball, M. C. (2000). Protection and storage of chlorophyll in overwintering evergreens. Proc Natl Acad Sci USA 97: 11098-11101.
Grzyb, J., Latowski, D. and Strzalka, K. (2006). Lipocalins—a family portrait. J Plant Physiol 163: 895-915.
Havaux, M., Dall′osto, L. and Bassi, R. (2007). Zeaxanthin has enhanced antioxidant capacity with respect to all other xanthophylls in Arabidopsis leaves and functions independent of binding to PSII antennae. Plant Physiol 145: 1506-1520.
Hernández-Gras, F. and Boronat, A. (2015). A hydrophobic proline-rich motif is involved in the intracellular targeting of temperature-induced lipocalin. Plant Molecular Biology 88: 301-311.
Hirono, Y. and Redei, G. P. (1963). Multiple Allelic Control of Chlorophyll b Level in Arabidopsis thaliana. Nature 197: 1324-1325.
Horton, P., Ruban, A. V. and Walters, R. G. (1996). Regulation of Light Harvesting in Green Plants. Annu Rev Plant Physiol Plant Mol Biol 47: 655-684.
Johnson, M. P. and Ruban, A. V. (2011). Restoration of rapidly reversible photoprotective energy dissipation in the absence of PsbS protein by enhanced DeltapH. J Biol Chem 286: 19973-19981.
Kim, E. H., Li, X. P., Razeghifard, R., Anderson, J. M., Niyogi, K. K., Pogson, B. J. and Chow, W. S. (2009). The multiple roles of light-harvesting chlorophyll a/b-protein complexes define structure and optimize function of Arabidopsis chloroplasts: a study using two chlorophyll b-less mutants. Biochim Biophys Acta 1787: 973-984.
Krause, G., Vernotte, C. and Briantais, J.-M. (1982). Photoinduced quenching of chlorophyll fluorescence in intact chloroplasts and algae. Resolution into two components. Biochimica et Biophysica Acta (BBA)-Bioenergetics 679: 116-124.
Krause, G. H. (1988). Photoinhibition of photosynthesis. An evaluation of damaging and protective mechanisms. Physiologia Plantarum 74: 566-574.
Lambrev, P. H., Miloslavina, Y., Jahns, P. and Holzwarth, A. R. (2012). On the relationship between non-photochemical quenching and photoprotection of Photosystem II. Biochim Biophys Acta 1817: 760-769.
Levesque-Tremblay, G., Havaux, M. and Ouellet, F. (2009). The chloroplastic lipocalin AtCHL prevents lipid peroxidation and protects Arabidopsis against oxidative stress. Plant J 60: 691-702.
Li, X. P., Björkman, O., Shih, C., Grossman, A. R., Rosenquist, M., Jansson, S. and Niyogi, K. K. (2000). A pigment-binding protein essential for regulation of photosynthetic light harvesting. Nature 403: 391-395.
Li, Z., Ahn, T. K., Avenson, T. J., Ballottari, M., Cruz, J. A., Kramer, D. M., Bassi, R., Fleming, G. R., Keasling, J. D. and Niyogi, K. K. (2009a). Lutein accumulation in the absence of zeaxanthin restores nonphotochemical quenching in the Arabidopsis thaliana npq1 mutant. Plant Cell 21: 1798-1812.
Li, Z., Wakao, S., Fischer, B. B. and Niyogi, K. K. (2009b). Sensing and responding to excess light. Annu Rev Plant Biol 60: 239-260.
Müller, P., Li, X. P. and Niyogi, K. K. (2001). Non-photochemical quenching. A response to excess light energy. Plant Physiol 125: 1558-1566.
Müller-Moulé, P., Conklin, P. L. and Niyogi, K. K. (2002). Ascorbate deficiency can limit violaxanthin de-epoxidase activity in vivo. Plant Physiol 128: 970-977.
Neff, M. M., Turk, E. and Kalishman, M. (2002). Web-based primer design for single nucleotide polymorphism analysis. Trends Genet 18: 613-615.
Nilkens, M., Kress, E., Lambrev, P., Miloslavina, Y., Muller, M., Holzwarth, A. R. and Jahns, P. (2010). Identification of a slowly inducible zeaxanthin-dependent component of non-photochemical quenching of chlorophyll fluorescence generated under steady-state conditions in Arabidopsis. Biochim Biophys Acta 1797: 466-475.
Niyogi, K. K., Bjorkman, O. and Grossman, A. R. (1997). The roles of specific xanthophylls in photoprotection. Proc Natl Acad Sci USA 94: 14162-14167.
Niyogi, K. K., Grossman, A. R. and Bjorkman, O. (1998). Arabidopsis mutants define a central role for the xanthophyll cycle in the regulation of photosynthetic energy conversion. Plant Cell 10: 1121-1134.
Noctor, G., Mhamdi, A. and Foyer, C. H. (2014). The roles of reactive oxygen metabolism in drought: not so cut and dried. Plant Physiol 164: 1636-1648.
Öquist, G. and Huner, N. P. (2003). Photosynthesis of overwintering evergreen plants. Annual Review of Plant Biology 54: 329-355.
Oster, U., Tanaka, R., Tanaka, A. and Rudiger, W. (2000). Cloning and functional expression of the gene encoding the key enzyme for chlorophyll b biosynthesis (CAO) from Arabidopsis thaliana. Plant J 21: 305-310.
Pogson, B., McDonald, K. A., Truong, M., Britton, G. and DellaPenna, D. (1996). Arabidopsis carotenoid mutants demonstrate that lutein is not essential for photosynthesis in higher plants. Plant Cell 8: 1627-1639.
Ramel, F., Ksas, B., Akkari, E., Mialoundama, A. S., Monnet, F., Krieger-Liszkay, A., Ravanat, J. L., Mueller, M. J., Bouvier, F. and Havaux, M. (2013). Light-induced acclimation of the Arabidopsis chlorina1 mutant to singlet oxygen. Plant Cell 25: 1445-1462.
Roach, T. and Krieger-Liszkay, A. (2012). The role of the PsbS protein in the protection of photosystems I and II against high light in Arabidopsis thaliana. Biochimica et Biophysica Acta (BBA)-Bioenergetics 1817: 2158-2165.
Ruban, A. V. (2016). Non-photochemical chlorophyll fluorescence quenching: mechanism and effectiveness in protection against photodamage. Plant Physiol.
Schaller, S., Latowski, D., Jemiola-Rzeminska, M., Dawood, A., Wilhelm, C., Strzalka, K. and Goss, R. (2011). Regulation of LHCII aggregation by different thylakoid membrane lipids. Biochim Biophys Acta 1807: 326-335.
Schneeberger, K., Ossowski, S., Lanz, C., Juul, T., Petersen, A. H., Nielsen, K. L., Jorgensen, J. E., Weigel, D. and Andersen, S. U. (2009). SHOREmap: simultaneous mapping and mutation identification by deep sequencing. Nat Methods 6: 550-551.
Sorek, N., Szemenyei, H., Sorek, H., Landers, A., Knight, H., Bauer, S., Wemmer, D. E. and Somerville, C. R. (2015). Identification of MEDIATOR16 as the Arabidopsis COBRA suppressor MONGOOSEL Proc Natl Acad Sci USA 112: 16048-16053.
Sylak-Glassman, E. J., Malnoë, A., De Re, E., Brooks, M. D., Fischer, A. L., Niyogi, K. K. and Fleming, G. R. (2014). Distinct roles of the photosystem II protein PsbS and zeaxanthin in the regulation of light harvesting in plants revealed by fluorescence lifetime snapshots. Proc Natl Acad Sci USA 111: 17498-17503.
Takabayashi, A., Kurihara, K., Kuwano, M., Kasahara, Y., Tanaka, R. and Tanaka, A. (2011). The Oligomeric States of the Photosystems and the Light-Harvesting Complexes in the Chl b-Less Mutant. Plant and Cell Physiology 52: 2103-2114.
Tanaka, A., Ito, H., Tanaka, R., Tanaka, N. K., Yoshida, K. and Okada, K. (1998). Chlorophyll a oxygenase (CAO) is involved in chlorophyll b formation from chlorophyll a. Proc Natl Acad Sci USA 95: 12719-12723.
Tardif, G., Kane, N. A., Adam, H., Labrie, L., Major, G., Gulick, P., Sarhan, F. and Laliberte, J. F. (2007). Interaction network of proteins associated with abiotic stress response and development in wheat. Plant Mol Biol 63: 703-718.
Tomitani, A., Okada, K., Miyashita, H., Matthijs, H. C., Ohno, T. and Tanaka, A. (1999). Chlorophyll b and phycobilins in the common ancestor of cyanobacteria and chloroplasts. Nature 400: 159-162.
Tsugama, D., Liu, S. and Takano, T. (2011). A rapid chemical method for lysing Arabidopsis cells for protein analysis. Plant Methods 7: 22.
Verhoeven, A. S., Adams, I. W., Demmig-Adams, B., Croce, R. and Bassi, R. (1999). Xanthophyll cycle pigment localization and dynamics during exposure to low temperatures and light stress in Vinca major. Plant Physiol 120: 727-738.
Walters, R. G. and Horton, P. (1993). Theoretical assessment of alternative mechanisms for non-photochemical quenching of PS II fluorescence in barley leaves. Photosynth Res 36: 119-139.
Weigel, D. and Glazebrook, J. (2006). Setting Up Arabidopsis Crosses. Cold Spring Harbor Protocols 2006: pdb.prot4623.
Woo, J. W., Kim, J., Kwon, S. I., Corvalan, C., Cho, S. W., Kim, H., Kim, S.-G., Kim, S.-T., Choe, S. and Kim, J.-S. (2015). DNA-free genome editing in plants with preassembled CRISPR-Cas9 ribonucleoproteins. Nat Biotech 33: 1162-1164.

### Example 1

### The Chloroplastic Lipocalin is Involved in a Sustained Photoprotective Mechanism Regulated by the Suppressor of Quenching 1 Protein in Arabidopsis thaliana

Nonphotochemical quenching (NPQ) comprises mechanisms by which photosynthetic organisms harmlessly dissipate excess absorbed light energy. Photoinhibitory quenching (qI), thought to be the result of photoinactivation of PSII, is the slowest component of NPQ to reverse and is the least understood. The possibility that part of qI may be photoprotective has been little examined, in part because of the lack of mutants directly affecting qI.

In the model plant Arabidopsis thaliana, the soq1 mutant displays additional slowly reversible NPQ relative to wild type. To identify molecular players of this NPQ pathway, we screened for suppressors of soq1 that showed a low level of NPQ, and mutants affecting either chlorophyllide a oxygenase (CAO) or the chloroplastic lipocalin protein (CHL) were isolated. Mutants affecting CAO are devoid of oligomerized PSII peripheral antenna proteins (LHCII), strongly suggesting that the additional quenching observed in soq1 occurs in LHCII. Because lipocalins are proteins that bind small hydrophobic molecules, we hypothesize that the quenching in soq1 stems from a modification to a hydrophobic molecule. We found that the CHL-dependent NPQ mechanism operates under stress conditions such as cold and high light, and our results suggest that SOQ1 inhibits CHL dependent quenching under non-stress conditions. We propose that, under stress conditions, CHL protects the thylakoid membrane by either forming quenching sites in the antenna of PSII, thereby preventing singlet oxygen stress or detoxify peroxidized lipids which in turn allow photoprotective quenching to occur.

Chlorophyllide a Oxygenase (CAO) Suppressors Identify the Peripheral Antenna of PSII as the Site of SOQ1-Related Quenching.

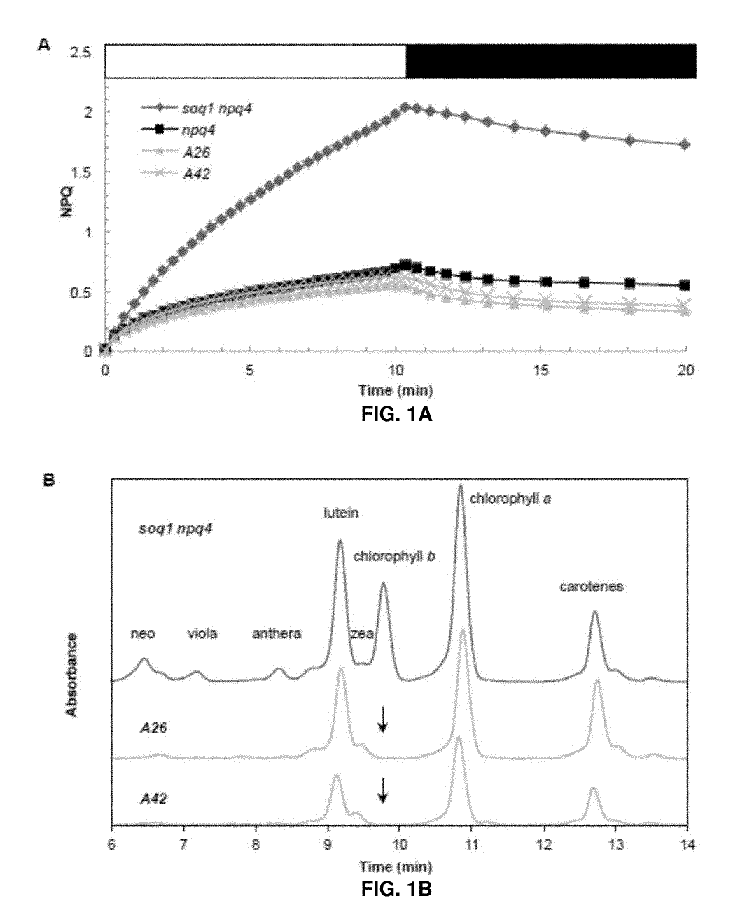

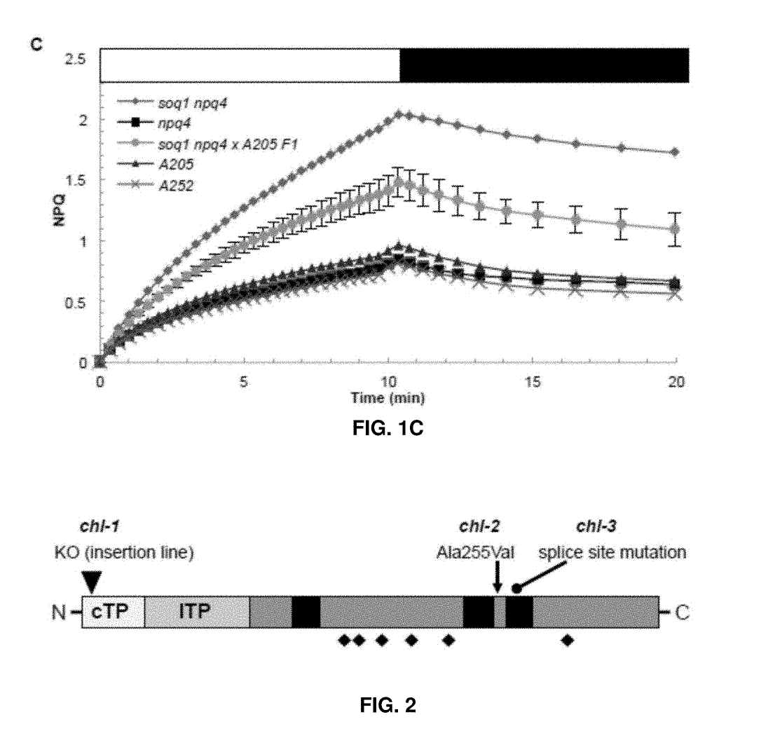

To elucidate the SOQ1-related quenching mechanism further, we conducted a suppressor screen using the soq1 npq4 mutant. We chose this double mutant as the starting strain for ease of identification of suppressors (NPQ phenotype returning to the level of npq4 from soq1 npq4 instead of returning to the level of the Col-0 wild type from soq1) and to minimize identification of mutations affecting the PsbS-dependent qE or ΔpH formation. An ethyl methanesulfonate (EMS)-mutagenized M2 population was generated and screened by video imaging of chlorophyll a fluorescence for suppression of the additional, slowly reversible quenching. Out of 22,000 mutant individuals screened, a class comprised of two independent mutants, A26 and A42, showed a “pale green” phenotype and displayed NPQ kinetics similar to that of npq4 (FIG. 1A). HPLC analysis of pigments showed that the visible pale green phenotype was due to lack of chlorophyll b (FIG. 1B). This pigmentation phenotype has previously been observed in mutants defective in the gene encoding chlorophyllide a oxygenase (CAO), which is required for chlorophyll b synthesis (Espineda et al., 1999). Sequencing the CAO gene in mutants A26 and A42 revealed single base pair (C-to-T) mutations, resulting in T375I and Q89STOP, respectively. As three mutant CAO alleles have been previously described in Arabidopsis (Hirono and Redei 1963, Espineda et al., 1999, Oster et al., 2000), we named these new alleles chlorina1-4 and chlorina1-5, respectively.

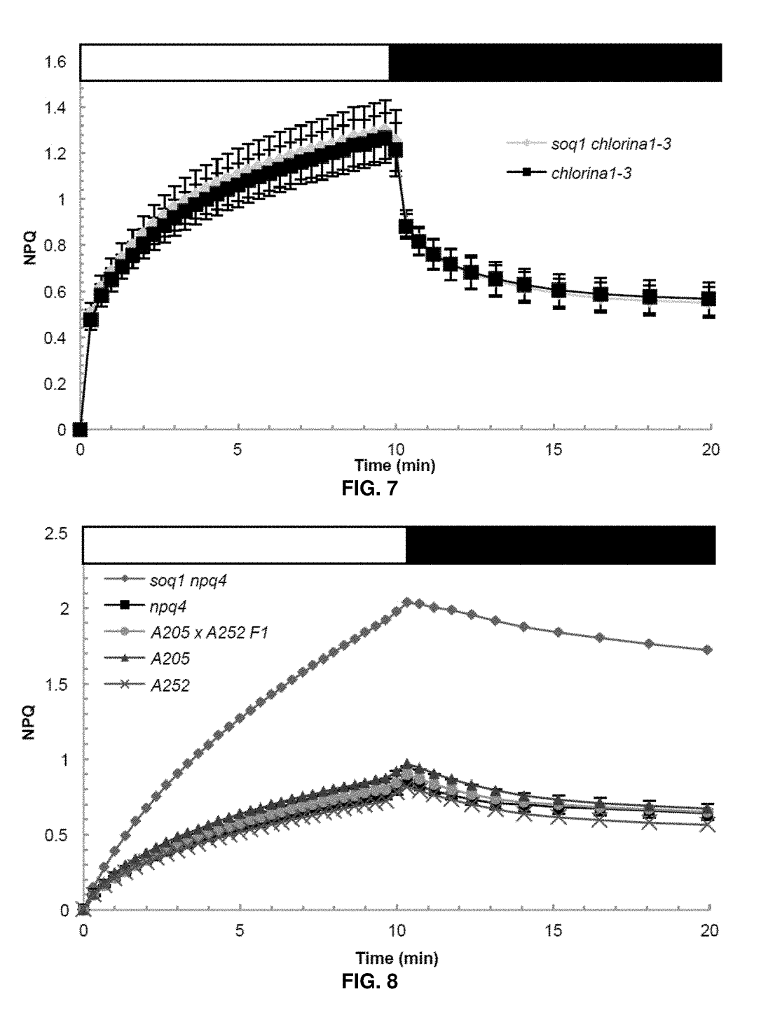

The null alleles (chlorina1-1 and -3) have been shown previously to be devoid of oligomeric organization of Lhcb proteins such as trimeric LHCII and PSII-LHCII supercomplexes but to still accumulate apo-monomeric Lhcb proteins (not containing chlorophyll) and monomeric Lhcb containing chlorophyll a (Espineda et al., 1999, Havaux et al., 2007, Takabayashi et al., 2011). Analysis of progeny of a cross between soq1 and the chlorina1-3 mutant confirmed that additional NPQ depends on the presence of chlorophyll b and oligomeric structure of PSII peripheral antenna proteins (FIG. 7). In this experiment, we grew chlorina1-3 and soq1 chlorina1-3 in very low light conditions (20 μmol photons m⁻² s⁻¹) and induced NPQ with very high light intensity (2000 μmol photons m⁻² s⁻¹) to ensure that enough light was absorbed to induce NPQ and that the difference between growth light and NPQ induction light intensities was large enough. The soq1 chlorina1-3 double mutant did not show additional quenching compared to chlorina1-3 (FIG. 7).

A Class of Suppressors from the Genetic Screen that does not Show a Pigment Defect.

Another class comprised of two independent mutants, A205 and A252, showed a “normal green” phenotype and displayed NPQ kinetics similar to that of npq4 (FIG. 1C). The F1 progenies of a cross between these two suppressor mutants (homozygous for soq1 and npq4 but heterozygous for each new mutation) showed a low level of NPQ similar to that of npq4 (FIG. 8), indicating that the two mutations belong in the same complementation group. We therefore proceeded to the genetic analysis of only one of these two mutants (A205). The mutation in A205 is semi-dominant as shown by the intermediate NPQ phenotype of the F1 progenies from the cross soq1 npq4×A205 (FIG. 1C). The low NPQ phenotype segregated in a 1:2:1 pattern in the F2 generation from this cross, indicating that the phenotype is caused by mutation of a single nuclear gene.

Identification of the Mutated Gene in “Normal Green” Suppressors Using Whole-Genome Sequencing.

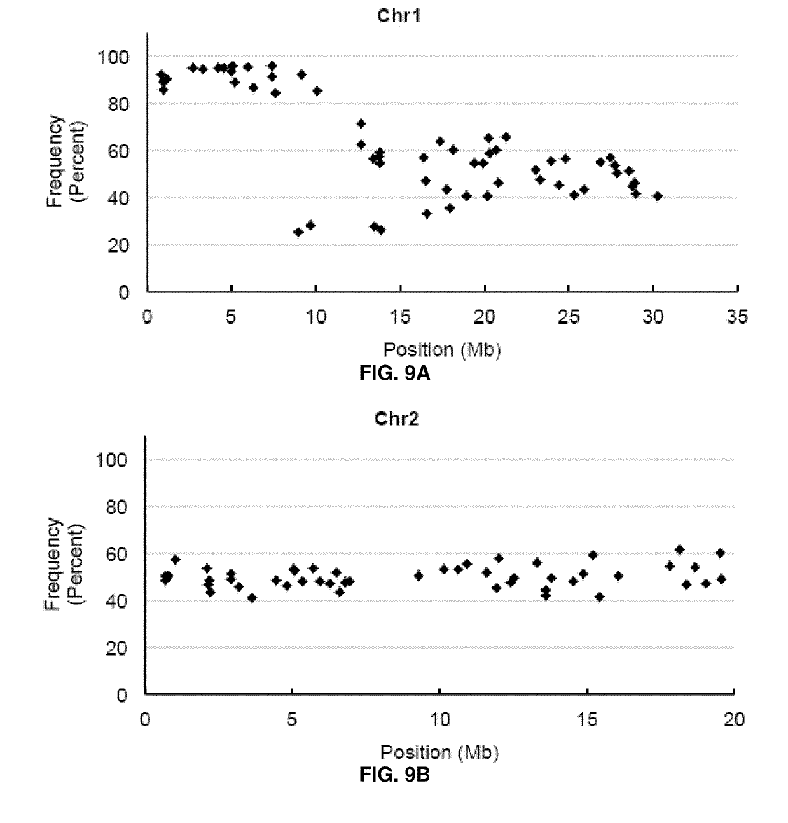

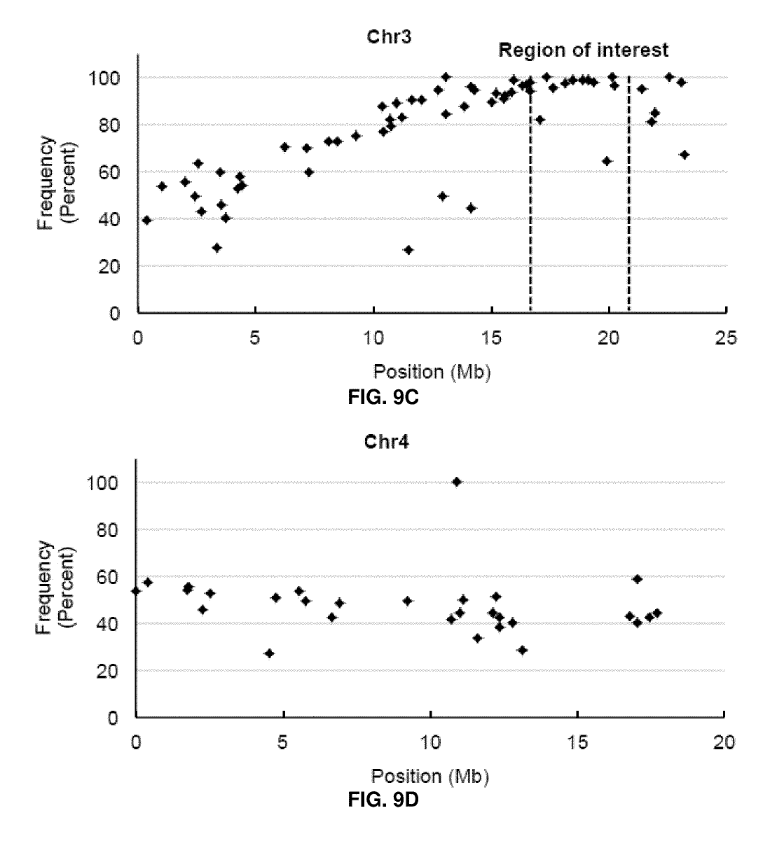

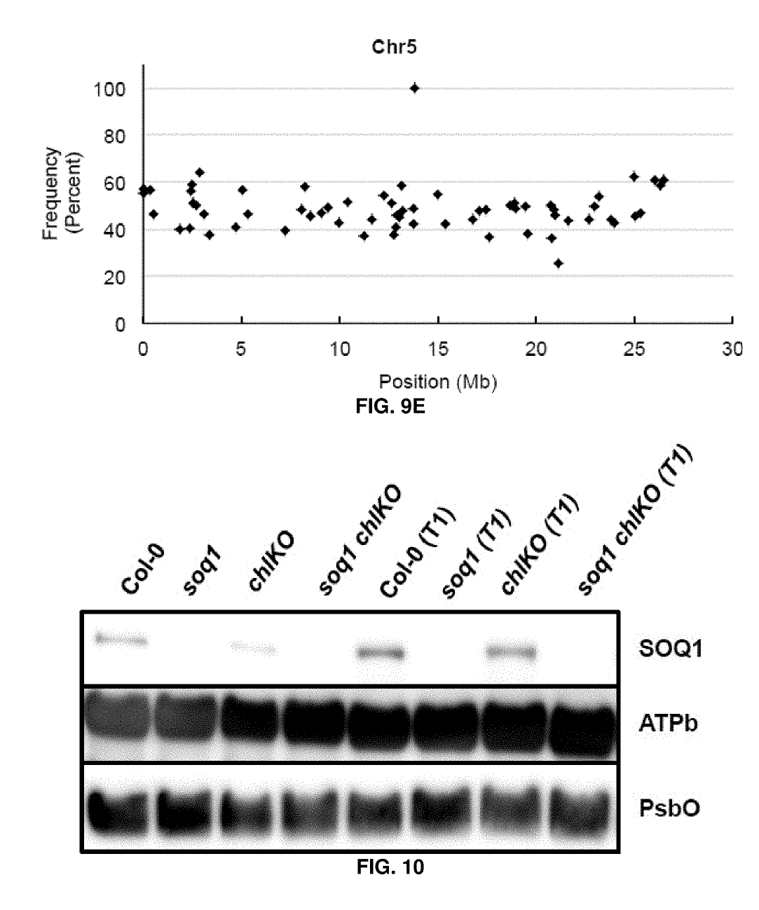

Mapping-by-sequencing in Arabidopsis has recently been proven successful in several studies (Schneeberger et al., 2009, Sorek et al., 2015) to determine the causative mutation of a specific phenotype. To this aim, we backcrossed the A205 mutant to the parental strain soq1 npq4 used for the EMS mutagenesis and selected individuals that showed low NPQ values, similar to the npq4 mutant, from the F2 progeny. Genomic DNA was extracted from a pool of 75 F2 seedlings exhibiting the mutant phenotype and subjected to whole-genome sequencing. The sequencing reads were mapped onto the Col-0 Arabidopsis reference genome with approximately 100× average coverage (Table 1), and single nucleotide polymorphisms were identified. The position and frequency of each single nucleotide polymorphism was plotted to look for a region of the genome showing enrichment in the allelic frequency of segregating mutations (FIGS. 9A to 9E). An increase in the allelic frequency of mutations approaching 100% was observed in the region between 16.5 and 21 Mbp on chromosome 3 (FIG. 9C), identifying this region as the one containing the causative mutation. We sequenced the A252 mutant, which contains an independent mutant allele of the gene of interest. Of the five genes containing mutations predicted to cause an amino acid change within the mapped region of A205, only one gene, At3g47860 encoding the chloroplastic lipocalin CHL, was also mutated in A252 (Table 2). Nucleotide transitions C to T and G to A resulted in A1a255Val in A205 and a mutated splice site in A252, respectively (FIG. 2). We named these new alleles chl-2 and chl-3 respectively and accordingly named the knockout allele of CHL (T-DNA SALK insertion line) studied in (Levesque-Tremblay et al., 2009) chl-1. We will use chlKO when referring to the latter line for clarity.

TABLE 1
Sequencing and read mapping summary.

| | Total Reads | Mapped Reads | Average Coverage |
|---|---|---|---|
| soq1 npq4 gl1 | 132,003,442 | 115,078,143 | 95.77 |
| A205 F2 | 155,298,686 | 134,982,882 | 112.36 |
| A252 M3 | 175,884,610 | 127,305,232 | 105.86 |

The samples were multiplexed and ran with an unrelated fourth sample in two lanes on an Illumina HiSeq2000/2500 to obtain 100 bp paired-end reads. The reads were mapped to the Col-0 reference sequence from TAIR.

TABLE 2
Summary of identified mutations within mapped region.

| | | | | | Gene |
|---|---|---|---|---|---|
| Chromo- | | Nucleotide | | AA | mutated |
| some | Position | Change | AGI | Change | in A252? |
| Chr3 | 16,691,263 | G−>A | At3g45510 | P156S | No |
| Chr3 | 17,657,162 | G−>A | At3g47860 | A255V | Yes |
| Chr3 | 18,481,261 | G−>A | At3g49820 | E9K | No |
| Chr3 | 19,132,895 | G−>A | At3g51580 | T41I | No |
| Chr3 | 20,168,663 | G−>A | At3g54470 | A383V | No |

Five mutations predicted to result in amino acid changes were identified within the mapped region (16.5-21 Mbp on chromosome 3) for the A205 mutant. Only one of these genes, At3g47860 encoding for the chloroplastic lipocalin CHL, was also disrupted in the allelic A252 mutant.

Chloroplastic Lipocalin (CHL) is Required for SOQ1-Related Quenching to Occur.

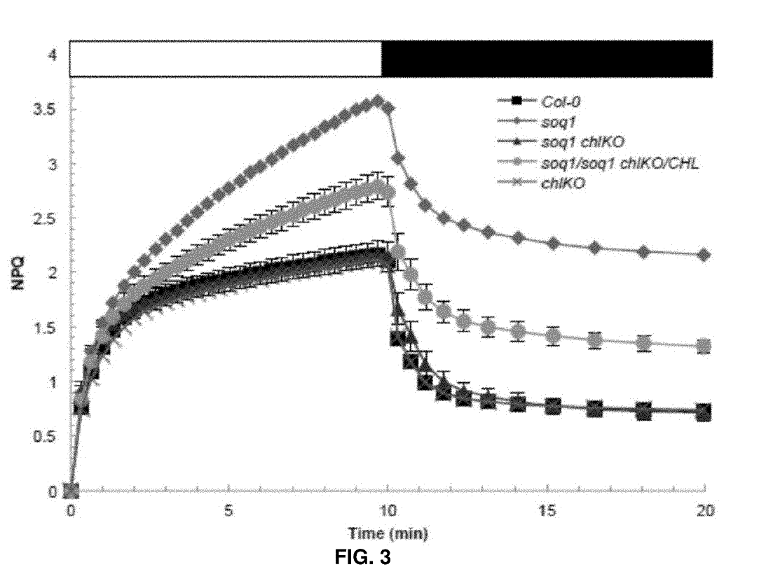

We examined the NPQ phenotype of chlKO as it had not been described previously. We found that chlKO exhibits the same NPQ kinetics and amplitude as wild type when grown under standard conditions and induced at 1200 μmol photons m⁻² s⁻¹. This result indicates that CHL does not have a role in NPQ under these conditions. However as was evidenced by the two suppressor mutants A205 and A252, the additional NPQ observed in the soq1 npq4 mutant relies on the CHL protein. To confirm the involvement of the CHL protein in the SOQ1-related quenching, we crossed the single soq1 mutant to the chlKO mutant allele. The soq1 chlKO double mutant shows an NPQ phenotype similar to wild type (FIG. 3), which further validates the requirement of the CHL protein for this quenching to occur. The soq1/soq1 chlKO/CHL strain shows NPQ kinetics intermediate to that of the soq1 mutant and the wild type, which means that the chlKO mutation is semi-dominant. Similarly, as stated above, the mutation CHL-A255V in chl-2 (A205) is semi-dominant in that the NPQ phenotype of the soq1/soq1 npq4/npq4 chl-2/CHL strain is intermediate to that of the soq1 npq4 mutant and npq4 mutant (FIG. 1C). We will now refer to SOQ1-related quenching as CHL-dependent quenching. Furthermore we can infer from our previous work (Brooks et al., 2013) and the work presented here that SOQ1 would inhibit CHL function, either directly or indirectly, because SOQ1 can suppress this quenching.

Immunoblot Analysis Shows that CHL Mobility is Altered in Soq1 Mutant.

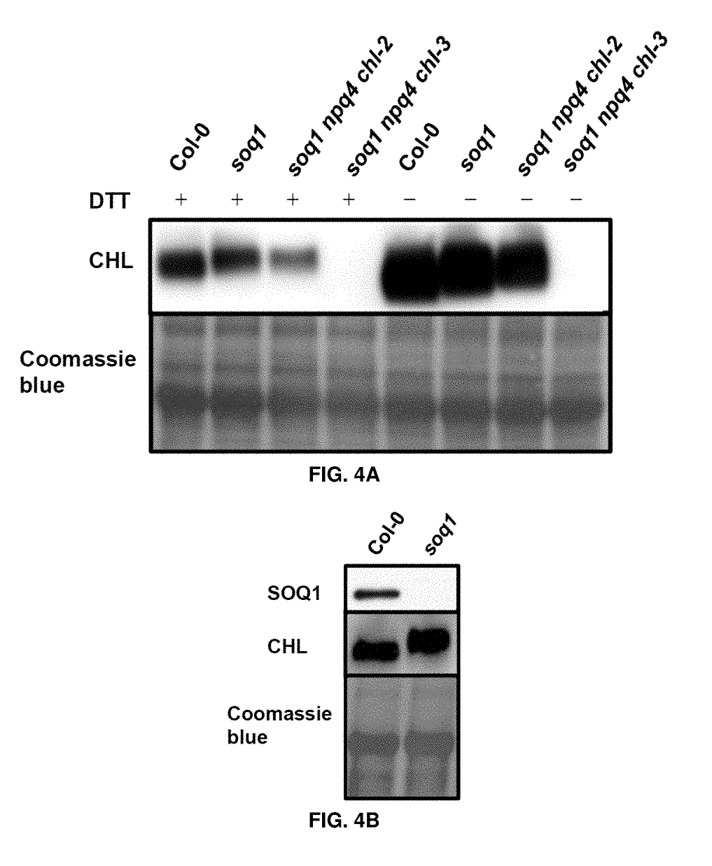

We probed the accumulation of the CHL protein in the suppressor mutants by immunoblot analysis. The amino acid change in the chl-2 (A205) mutant results in a lower accumulation of the protein (FIG. 4A). The fact that no additional quenching was observed in the soq1 npq4 mutant background together with this mutation (FIG. 1C, A205), although some of the protein is present in this mutant, suggests that the A1a255Val substitution renders the protein non-functional. The mutated splice site in chl-3 (A252) results in absence of the CHL protein (FIG. 4A). Interestingly, the apparent molecular mass of CHL is slightly higher in the soq1 mutant background (FIGS. 4A and B). This migration shift is also observed in the chl-2 (A205) mutant.

Because SOQ1 contains a thioredoxin-like domain in the lumen, it is possible that SOQ1 maintains its target(s) in a reduced state (Brooks et al., 2013). CHL contains six conserved cysteine residues (FIG. 2), so we tested whether the altered mobility of CHL in soq1 mutant background was an oxidized form of CHL. Addition of a reducing agent (DTT) did not however reverse this altered mobility (FIG. 4A). We tested whether this migration shift in the soq1 mutant required exposure to light, but it did not as this shift was still present if plants were kept in the dark for 14 h (FIG. 4B). Attempts to determine the reason for this altered mobility have been so far unsuccessful. This altered mobility importantly underlines the potential biochemical interaction, direct or indirect, between SOQ1 and CHL.

CHL-Dependent Quenching Operates in Chilling High Light Conditions.

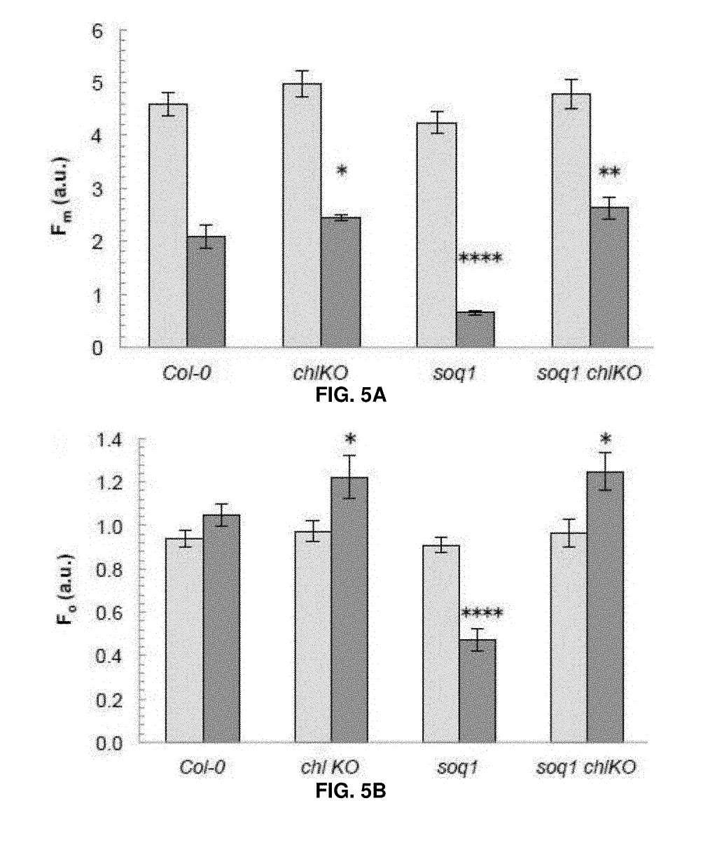

Both CHL mRNA and protein expression increase during abiotic stresses such as high light and drought (Levesque-Tremblay et al., 2009). Interestingly, the chlKO mutant shows increased lipid peroxidation after a high light (1300 μmol photons m⁻² s⁻¹) and cold treatment (7° C.) for 24 h (Levesque-Tremblay et al., 2009). We hypothesized that the CHL-dependent quenching contributes to abiotic stress resistance and tested induction of this quenching under chilling and high light conditions in the different genotypes (Col-0, chlKO, soq1, and soq1 chlKO). Under control conditions the three mutant genotypes displayed similar Fₘ values relative to Col-0 (FIG. 5A, light grey bars and Table 3A). After 8 h of cold (12° C.) and high light (1070 μmol photons m⁻² s⁻¹), Fₘ values for all genotypes decreased, indicating that quenching was induced by this treatment, but the decrease in Fₘ for chlKO and soq1 chlKO was consistently smaller than that of Col-0. This small but significant difference in Fₘ in plants lacking CHL represents the quenching conferred by CHL (FIG. 5A, dark grey bars and Table 3B). In addition, soq1 displayed a larger decrease in Fₘ compared to wild type, which reveals the contribution of CHL-dependent quenching during stress condition when its inhibitor SOQ1 is no longer regulating CHL activity.

TABLE 3
Output from Tukey's multiple comparisons test from one
way ANOVA performed on maximal fluorescence, Fm, values.

| | | | | |
|---|---|---|---|---|
| Tukey's multiple | Mean | | | Adjusted |
| comparisons test | Diff. | Significant? | Summary | P Value |

A

| | | | | |
|---|---|---|---|---|
| Col-0 vs. chlKO | −0.38 | No | ns | 0.1696 |
| Col-0 vs. soq1 | 0.35 | No | ns | 0.2217 |
| Col-0 vs. soq1 chlKO | −0.19 | No | ns | 0.7043 |
| chlKO vs. soq1 | 0.74 | Yes | ** | 0.005 |
| chlKO vs. soq1 chlKO | 0.20 | No | ns | 0.6693 |
| soq1 vs. soq1 chlKO | −0.54 | Yes | * | 0.037 |

B

| | | | | |
|---|---|---|---|---|
| Col-0 vs. chlKO | −0.35 | Yes | * | 0.0298 |
| Col-0 vs. soq1 | 1.43 | Yes | **** | <0.0001 |
| Col-0 vs. soq1 chlKO | −0.55 | Yes | ** | 0.0013 |
| chlKO vs. soq1 | 1.78 | Yes | **** | <0.0001 |
| chlKO vs. soq1 chlKO | −0.20 | No | ns | 0.3119 |
| soq1 vs. soq1 chlKO | −1.98 | Yes | **** | <0.0001 |

One-way ANOVA followed by Tukey's post hoc test was performed on fluorescence amplitudes measured with a PAM fluorimeter on leaf discs of same area from plants grown at 120 μmol photons m−2 s−1, 21° C. (A, control condition) and after a cold and high light treatment for 8 h at 1070 μmol photons m−2 s−1, 12° C. (B, treatment). Leaf discs from n = 4 individuals of each genotype were dark-acclimated for 40 min (on moist surface) to fully relax qE and part of qZ prior to measurement. “No” means there is no significant difference between the two genotypes tested for the fluorescence phenotype measured and “Yes” means there is a significant difference.

Similarly, after the chilling and high light treatment soq1 also displayed a large decrease in Fₒ, characteristic of the CHL-dependent antenna quenching (FIG. 5B). This strong quenching of Fₒ is not due to a damaged oxygen-evolving complex at PSII as evidenced by equivalent amounts of PsbO protein (FIG. 10). To further explain the stress sensitivity exhibited by the chlKO mutant under chilling high light conditions (Levesque-Tremblay et al., 2009), we tested whether operation of this quenching is photoprotective. FIG. 5B shows that Fₒ is higher in chlKO and soq1 chlKO mutants after cold and high light treatment compared to Col-0 and soq1 and pretreatment (see also Table 4), which is indicative of damage to PSII reaction centers (Aro et al., 1993). This result suggests that the CHL-dependent quenching mechanism is photoprotective.

TABLE 4
Output from Tukey's multiple comparisons test from one
way ANOVA performed on initial fluorescence, Fo, values.

| | | | | |
|---|---|---|---|---|
| Tukey's multiple | Mean | | | Adjusted |
| comparisons test | Diff. | Significant? | Summary | P Value |

A

| | | | | |
|---|---|---|---|---|
| Col-0 vs. chlKO | −0.03 | No | ns | 0.7432 |
| Col-0 vs. soq1 | 0.03 | No | ns | 0.8016 |
| Col-0 vs. soq1 chlKO | −0.03 | No | ns | 0.8736 |
| chlKO vs. soq1 | 0.07 | No | ns | 0.2693 |
| chlKO vs. soq1 chlKO | 0.01 | No | ns | 0.9933 |
| soq1 vs. soq1 chlKO | −0.06 | No | ns | 0.3844 |

B

| | | | | |
|---|---|---|---|---|
| Col-0 vs. chlKO | −0.17 | Yes | * | 0.0315 |
| Col-0 vs. soq1 | 0.58 | Yes | **** | <0.0001 |
| Col-0 vs. soq1 chlKO | −0.20 | Yes | * | 0.0133 |
| chlKO vs. soq1 | 0.75 | Yes | **** | <0.0001 |
| chlKO vs. soq1 chlKO | −0.03 | No | ns | 0.9589 |
| soq1 vs. soq1 chlKO | −0.78 | Yes | **** | <0.0001 |

One-way ANOVA followed by Tukey's post hoc test was performed on fluorescence amplitudes measured with a PAM fluorimeter on leaf discs of same area from plants grown at 120 μmol photons m−2 s−1, 21° C. (A, control condition) and after a cold and high light treatment for 8 h at 1070 μmol photons m−2 s−1, 12° C. (B, treatment). Leaf discs from n = 4 individuals of each genotype were dark-acclimated for 40 min (on moist surface) to fully relax qE and part of qZ prior to measurement. “No” means there is no significant difference between the two genotypes tested for the fluorescence phenotype measured and “Yes” means there is a significant difference.

### DISCUSSION

Slowly relaxing NPQ mechanisms have been grouped under the term qI for photoinhibitory quenching in which PSII inactivation is thought to be the result of photodamage, specifically to the D1 protein (Edelman and Mattoo 2008). Several lines of evidence suggest that not all of qI is due to photoinhibition (Demmig and Björkman 1987, Horton et al., 1996). We have previously identified the soq1 mutant that shows a form of sustained NPQ unrelated to PSII photodamage (Brooks et al., 2013). To identify the molecular partners involved in this slowly relaxing NPQ mechanism, we mutated the soq1 npq4 mutant and screened for suppressors that no longer exhibit this type of quenching.

SOQ1-Related NPQ Mechanism Depends on CHL.

We found that the chloroplastic lipocalin, CHL, is necessary for the additional quenching observed in the soq1 mutant, because neither the triple mutants soq1 npq4 chl-2 (A205) and soq1 npq4 chl-3 (A252) (FIG. 1C) nor the double mutant soq1 chlKO (FIG. 3) showed additional quenching. CHL has been previously shown to accumulate under drought and high light stresses where it is thought to function in preventing or modulating singlet oxygen (^{I}O₂)-mediated lipid peroxidation (Levesque-Tremblay et al., 2009). We investigated whether the stress sensitivity exhibited by the chlKO mutant after a cold and high light stress (Levesque-Tremblay et al., 2009) was due to the lack of the quenching mechanism executed by CHL. We indeed found that chilling and high light conditions results in sustained quenching of Fₘ that is CHL dependent (FIG. 5A and Table 3), indicating that the CHL-dependent quenching mechanism operates in wild type under these conditions. CHL-dependent quenching is equivalent to the difference in Fₘ depression between wild type and chlKO. Furthermore, operation of this quenching seems to be photoprotective, as shown by the increased Fₒ in mutants lacking CHL-dependent quenching when treated with chilling and high light (FIG. 5B and Table 4). Levesque-Tremblay et al. (2009) showed that chlKO stress sensitivity translated into a higher accumulation of lipid peroxidation after a cold and high light treatment and proposed a function for CHL in managing peroxidized lipids by either detoxification or prevention. NPQ has been proposed to mitigate ^{I}O₂ production (Müller et al., 2001) and PsbS-dependent quenching has been shown to limit ^{I}O₂ production (Roach and Krieger-Liszkay 2012). Our study therefore suggests that the accumulation of peroxidized lipids observed in chlKO following abiotic stress would be a consequence of the absence of the photoprotective NPQ mechanism operated by CHL and thus, that CHL would function in preventing the formation of peroxidized lipids.

Function of CHL in NPQ.

Lipocalins have great functional diversity (Flower 1996). The name lipocalin comes from the eight-stranded anti-parallel beta sheet that forms a barrel or a calyx (cup-like structure) and their high affinity for small hydrophobic molecules. A distinction is made between true lipocalins and lipocalin-like proteins, based on the number of structurally conserved regions (SCR) they contain (Charron et al., 2005). CHL belongs to the group of true lipocalins as it contains three SCRs (FIG. 2). In the Arabidopsis genome (or other land plants), there is another true lipocalin, TIL for temperature-induced lipocalin (Frenette Charron et al., 2002), which is located in different cell membranes and organelles, depending on growth conditions (Charron et al., 2005, Hernández-Gras and Boronat 2015). TIL and CHL have been shown to play a role during abiotic stress and to have overlapping functions in protecting against lipid peroxidation (Boca et al., 2014), but their mechanism of action is unknown.

The first reported plant lipocalin-like proteins were VDE and ZEP, the xanthophyll cycle enzymes (Bugos et al., 1998). Interestingly, they also play an important role in photoprotection (Niyogi et al., 1998). Proteins from the lipocalin family have been shown to bind or carry hydrophobic molecules such as retinoids, fatty acids, steroids, odorants and pheromones or can have enzymatic activity, e.g. prostaglandin isomerase (Grzyb et al., 2006). It is not known to what ligand or substrate CHL and TIL may bind, or whether these proteins exhibit enzymatic activity. Further experiments that aim at determining the ligand or substrate of CHL will provide insights for understanding the quenching mechanism. Interestingly, heterozygotes for the mutation in the CHL gene in a soq1 homozygote context showed an intermediate NPQ phenotype (FIGS. 1C and 3). This semi-dominance could signify that CHL has an enzymatic activity and is rate-limiting for the reaction it catalyzes as was proposed for PsbS and LUT2 based on heterozygotes of npq4 (Li et al., 2000) and lut2 (Pogson et al., 1996), mutations that show a similar dosage-dependent phenotype. Formally, CHL could be the site of quenching itself, but its lumenal localization (even if tethered to the membrane during quenching activity) seems to be incompatible with a hypothesized charge- or energy-transfer from the PSII peripheral antenna (see below) to CHL in terms of distance. In either case, our results imply that there is a linear correlation between the magnitude of CHL-dependent NPQ and the amount (or activity) of CHL rather than this NPQ being limited by a putative substrate. The variant CHL-A255V shows a lower accumulation of the protein (FIG. 4A) and a complete loss of additional NPQ in the soq1 mutant context (FIG. 1C). Taken together with the semidominant nature of the chlKO mutation, we can conclude that A255V destabilizes the CHL protein and results in a loss of function, otherwise the lower accumulation of protein in soq1 npq4 chl-2 (A205) would permit some additional NPQ compared to soq1 npq4 chl-3 (A252). Residue A255 from AtCHL shows 100% conservation among the eight sequences of CHL homologs analyzed in (Charron et al., 2005) and is located at the end of SCR2 (FIG. 2), consistent with its likely indispensability for CHL function.

The Site of CHL-Dependent NPQ is in the Antenna of PSII.

In our suppressor screen on soq1 npq4, we also identified two new mutant alleles affecting chlorophyllide a oxygenase (CAO) as demonstrated by the absence of chlorophyll b in the mutants A26 and A42 (FIG. 1B) and confirmed by candidate gene sequencing. CAO is located in the chloroplast and catalyzes a two-step oxygenase reaction involved in the synthesis of chlorophyll b through its cofactors: a Rieske [2Fe2S cluster] and a non-heme iron (Tanaka et al., 1998). The alleles described until now in Arabidopsis were obtained by X-ray mutagenesis. chlorina1-1 is a null allele and accumulates a truncated form of the protein (415 amino acids out of 536). chlorina1-2 is a leaky allele and contains an amino acid change V274E within the 2Fe2S cluster binding site. chlorina1-3 is a null allele with a deletion of 40 amino acids at the iron-binding site. We have found here through EMS mutagenesis two additional alleles named chlorina1-4 and chlorina1-5, which respectively correspond to Q89STOP and T375I. The truncated protein resulting from the early stop codon in chlorina1-4 is likely to produce a nonfunctional protein. T375 is a conserved amino acid (Tomitani et al., 1999) located in the vicinity of the iron-binding site, suggesting that chlorina1-5 is likely to affect catalytic activity. As chlorophyll b was not detected in either chlorina1-4 or -5 (FIG. 1B), it appears that they are both null alleles of CAO, consistent with the nature of the mutations.

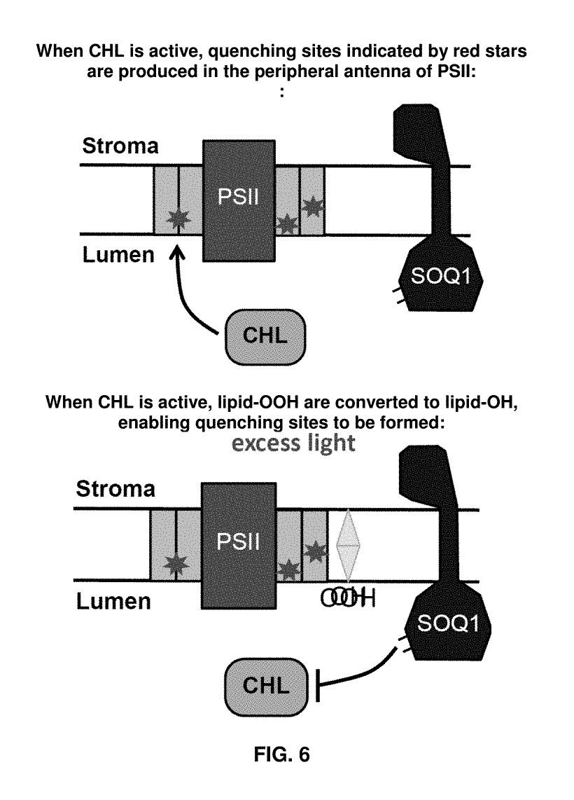

The soq1 npq4 chlorina1-4 and -5 mutants displayed a low level of NPQ similar to that of npq4 (FIG. 1A), and accordingly the soq1 chlorina1-3 displayed the same level of NPQ as the chlorina1-3 mutant (FIG. 7). A chlorina1 mutant lacks oligomeric organization of Lhcb proteins such as trimeric LHCII and PSII-LHCII supercomplexes but still accumulates apo-monomeric Lhcb proteins (not containing chlorophyll) and monomeric Lhcb containing chlorophyll a (Espineda et al., 1999, Havaux et al., 2007, Takabayashi et al., 2011). The absence of oligomeric PSII peripheral antenna in a soq1 mutant background abolishes induction of additional quenching, therefore we conclude that the CHL-dependent quenching mechanism occurs in the oligomeric peripheral antenna of PSII (FIG. 6). Future study will explore the specific antenna protein(s) that are necessary for CHL-dependent quenching. Lipid composition is known to modulate LHCII aggregation state and function (Schaller et al., 2011). It is possible that CHL-mediated modification of a hydrophobic molecule, such as a thylakoid membrane lipid, would change LHCII conformation and thus create a quenching site. Interestingly a potential biochemical interaction between the wheat CHL was found by yeast-two-hybrid (using dehydrated plant cDNA libraries) (Tardif et al., 2007) with the lipid transfer protein 3 (LTP3) and a β-ketoacyl-acyl carrier protein synthase involved in fatty acid synthesis. These potential interactions and their relevance for CHL function will need to be examined further.

Alternatively, because this quenching decreases Fₒ (FIG. 5B, soq1), it could be the result of photodamage at the donor side of PSII (oxygen-evolving complex), but similar accumulation of Psb0 in Col-0 and soq1 argues against this hypothesis (FIG. 10). Furthermore chlorina1 has been shown to be a^{I}O₂ overproducer (Ramel et al., 2013); we would thus expect more photodamage from low light grown plants in a chlorina1 mutant background during a 10 min illumination with high light and yet the soq1 chlorina1 mutant does not show any additional quenching (FIG. 7).

Regulation of CHL-Dependent Quenching Mechanism by SOQ1.

The suppressor screen revealed a genetic interaction between SOQ1 and CHL: upon mutation of CHL in a soq1 mutant background (soq1 chlKO), the additional quenching is no longer induced (FIG. 3). This result demonstrates that CHL is required for this quenching as discussed above and that the function of SOQ1 is to inhibit (quenching by) CHL (FIG. 6). Alternatively SOQ1 could be involved in removing or recycling the quenching sites formed by CHL, but we do not favor this idea for several reasons. As CHL is located in the thylakoid lumen (Levesque-Tremblay et al., 2009), it is a good candidate for interacting with the SOQ1 domains responsible for regulating this NPQ mechanism, namely the thioredoxin-like and NHL-beta propeller domains (Brooks et al., 2013). A biochemical interaction is also suggested by the altered mobility of CHL in the soq1 mutant, shown by immunoblot analysis (FIGS. 4A and 4B). This altered mobility is not affected by mutation A255V and is not reversed by addition of a reducing agent like DTT (FIG. 4A), which could mean that it is not a redox modification or that it is a stable modification such as cysteine sulfinic or sulfonic acid or oxidized methionine that cannot be reversed by DTT. The altered mobility form of CHL in the soq1 mutant does not return to the wild-type form after 14 hours in the dark (FIG. 4B), which suggests that SOQ1 is required to reverse this slowly migrating form and that it is not induced by light exposure. Whether the interaction between SOQ1 and CHL is direct or indirect will be tested in the future. Additionally, it is possible that this altered mobility form of CHL constitutes the active form of CHL or a form that covalently binds the ligand or substrate. The variant CHL-A255V also displays this altered mobility (FIG. 4A) and is inactive, but this inactivity is likely due to the impaired function from the amino acid change.

Under chilling and high light conditions, wild type did not show the similar extent of Fₘ and Fₒ quenching as the soq1 mutant (FIGS. 5A and B, and Tables 3 and 4). This result further shows how the SOQ1 protein inhibits CHL-dependent quenching in a wild-type context under these conditions, but the inhibition is only partial because wild type displays more quenching than a chlKO mutant. The SOQ1 gene has been reported to be downregulated during drought-stress as summarized by Noctor et al. (2014). This is a possible way to alleviate the inhibition of CHL during such abiotic stresses by repressing the inhibitor. However the soq1 mutation is recessive, which might mean that a low level of SOQ1 protein is sufficient for its function. This leads us to think that the repression of qI by SOQ1 might be more complex than a binary system in which less of the repressor means more active target and would constitute instead a way to fine-tune CHL function. CHL and SOQ1 genes are conserved among all land plants with sequenced genomes, so it is possible that this quenching mechanism is conserved among all land plants.

Physiological Relevance of a ΔpH-Independent Quenching Mechanism.

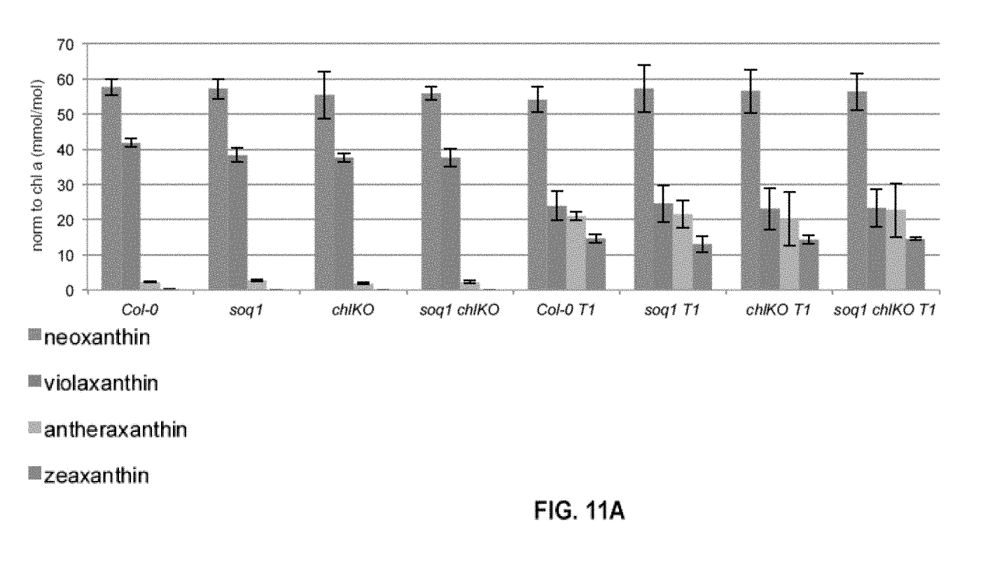

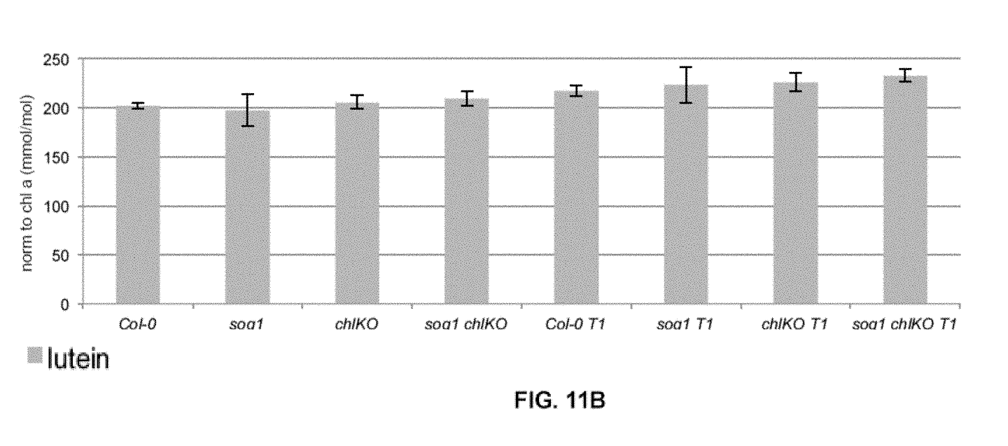

The CHL-dependent quenching mechanism does not depend on ΔpH, and this characteristic might provide a fitness advantage under specific environmental conditions. In Arabidopsis, we present evidence that this quenching is induced in wild type during chilling plus high light stress (FIGS. 5A and 5B). Previous research by Dall'Osto et al. (2005) provided evidence for a ΔpH-independent quenching mechanism in plants that was later termed qZ (Nilkens et al., 2010) because it relies on the presence of zeaxanthin. This mechanism is independent of PsbS and is based on the conformational change of (at least) the minor antenna CP26. In our study, during the chilling high light experiment, we observed a decrease in Fₘ in all genotypes regardless of the presence or absence of CHL (FIG. 5A). We measured the zeaxanthin content remaining after the 40 min dark-acclimation and found that a low level of zeaxanthin was remaining (FIGS. 11A and 11B). Dall'Osto et al. (2005) stated that the zeaxanthin effect in qZ is saturated at rather low concentration. We therefore hypothesize that this CHL-independent decrease in Fₘ is qZ.

Furthermore, Dall'Osto et al. (2005) have discussed that qZ could be responsible for part of the sustained ΔpH-independent quenching mechanism observed in overwintering evergreens (Verhoeven et al., 1999, Gilmore and Ball 2000). A highly efficient quenching is necessary to enable overwintering evergreens to withstand extended periods of high light and cold (Adams III et al., 2002, Öquist and Huner 2003). We have previously discussed (Brooks et al., 2013) the possibility that the SOQ1-related or CHL-dependent quenching mechanism described here plays a role in this sustained type of NPQ. Tropical evergreens have also been shown to induce a sustained form of NPQ upon transition from shade to high light (Demmig-Adams et al., 2006), and it is likely that many plants need sustained quenching mechanisms to survive periods of extended light stress (Demmig-Adams and Adams 2006). In the future, it would be interesting to test whether qZ or the CHL-dependent quenching is the dominant form of quenching in this sustained NPQ mode in other plant species. With the recent advances in gene editing technology in non-model organisms (Woo et al., 2015), knock-out of CHL in an evergreen species would be a direct way to test the contribution of CHL to this sustained quenching mode.

CHL-Dependent Quenching Occurs in Wild-Type in Cold and High Light.

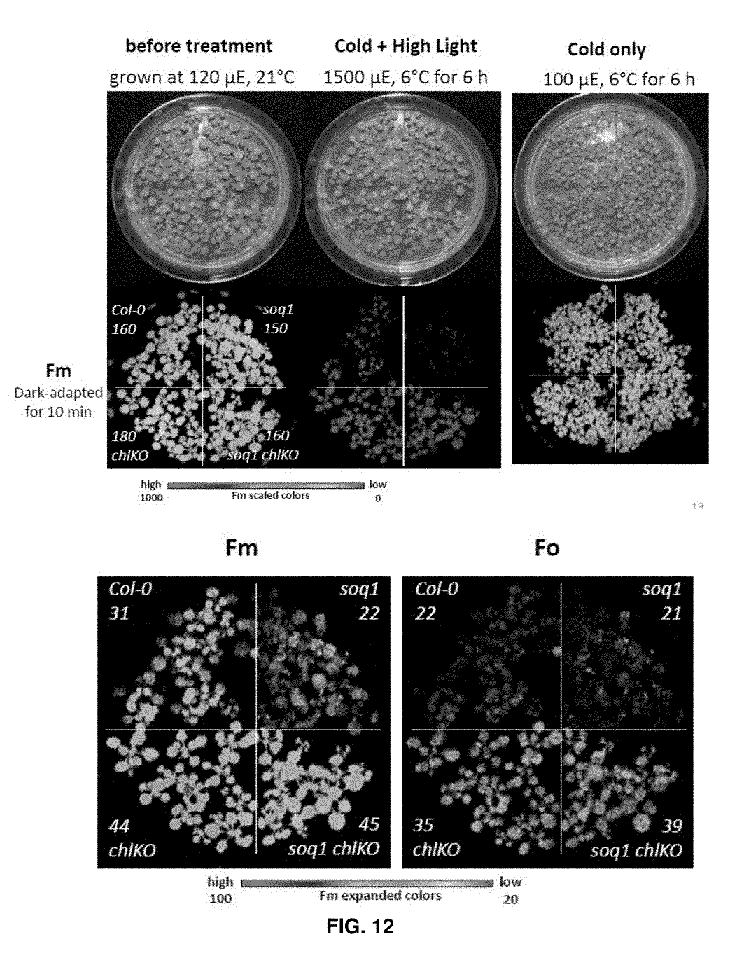

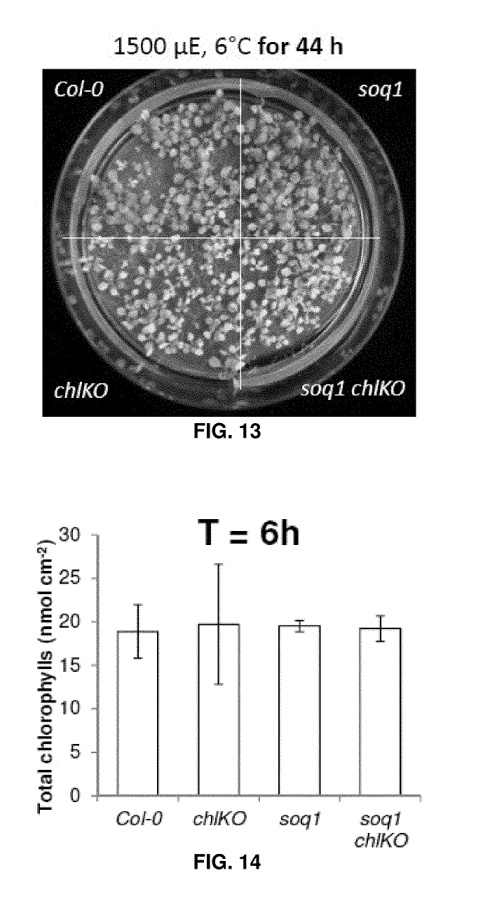

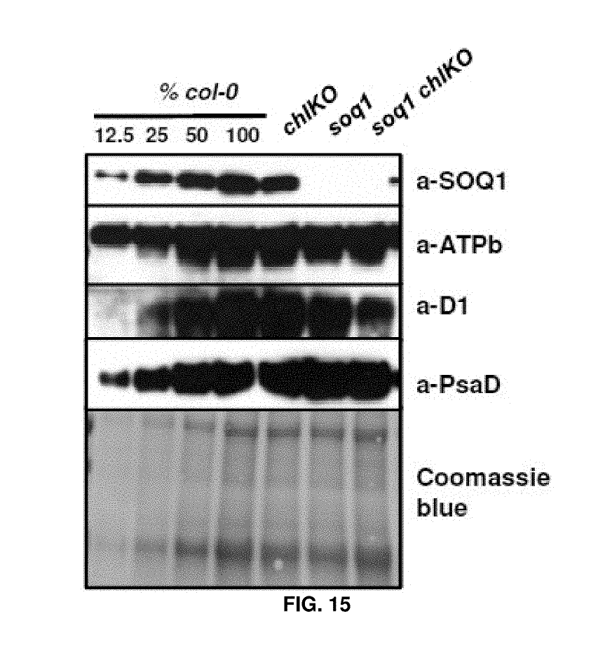

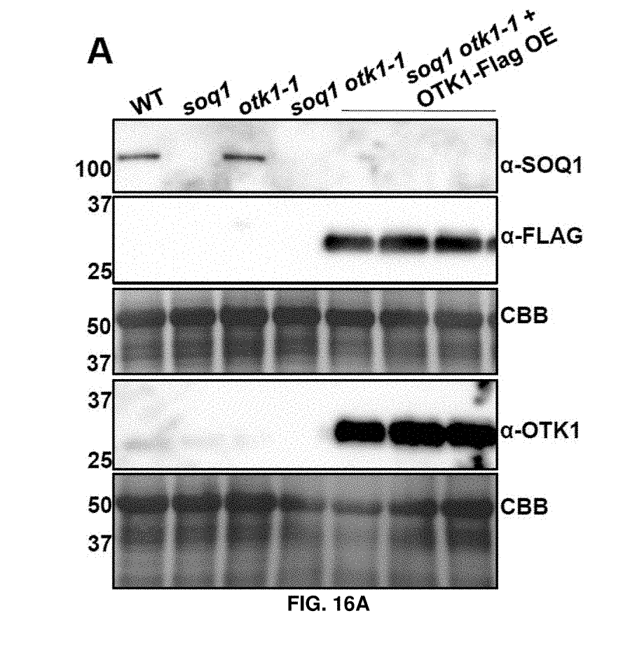

We hypothesize that the chlKO mutant exhibits stress sensitivity because it lacks antenna qI that is induced in HL and cold. There is possibly a direct involvement of CHL in forming quenching sites or indirect through conversion of lipid-OOH to lipid-OH (FIG. 6). We propose that SOQ1 inhibits, directly or indirectly, CHL activity under non-stress conditions. When CHL is active, quenching sites indicated by red stars are produced in the peripheral antenna of PSII. See FIGS. 12-15.

The chloroplastic lipocalin, CHL, has a role in photoprotection. The slow relaxing form of quenching, that relies on CHL, occurs in cold+HL. There is a dosage dependence of CHL for quenching amount. The quenching site is in the peripheral antenna of PSII. SOQ1 negatively regulates this quenching through, direct or indirect, modification of CHL. The soq1 mutation results in higher quenching in absence of lutein. When grown in high light, soq1 does not display additional quenching. Suppressor mutants display intermediate or altered NPQ kinetics between npq4 and soq1 npq4. Suppressor mutants D2 and A37 exhibit pigment defects (middle and right) compared to soq1 npq4 (1e4).

### Methods

Plant Material and Growth Conditions.

Wild-type Arabidopsis thaliana and derived mutants studied here are of Col-0 ecotype. Mutants npq4-1 (Li et al., 2000), soq1-1, soq1 npq4 glabrous (gl)1-1 (Brooks et al., 2013) were previously isolated in our laboratory. We will refer to the npq4-1 and soq1-1 mutant alleles as npq4 and soq1 respectively because no other mutant alleles of these genes were used in this study. Chlorina1 is usually abbreviated as chl. Because we also found mutations in the chloroplastic lipocalin abbreviated chl (Levesque-Tremblay et al., 2009), we decided to use the full name chlorina1 when referring to chl to avoid confusion. Mutant chlorina1-3 lhcb5 (Kim et al., 2009) was used as the source of the chlorina1-3 allele. Mutants soq1 npq4 gl1 chlorina1-4, soq1 npq4 gl1 chlorina1-5, soq1 npq4 gl1 chl-2, soq1 npq4 gl1 chl-3 were generated in this study. The chlKO T-DNA insertion line SALK 133049C was provided by F. Ouellet (Université du Québec à Montréal). Plants were grown on soil (Sunshine Mix 4/LA4 potting mix, Sun Gro Horticulture Distribution) under a 10/14 h light/dark photoperiod at 120 μmol photons m⁻² s⁻¹, unless stated otherwise, at 21° C. for 5 to 6 weeks or on agar plates containing 0.5×Murashige & Skoog medium (VWR Scientific 95026-314) at 100 μmol photons m⁻² s⁻¹ (continuous light) at 21° C. and then transferred to soil. For the cold and high light treatment, plants were placed for 8h at 1070 μmol photons m⁻² s⁻¹ and 12° C. Light bulbs used in growth chambers are cool white (4100K) from Philips (F25T8/TL841 25W) for plants grown on soil and from General Electric (F17T8/SP41 17W) for seedlings grown on agar plates.

Genetic Crosses and Genotyping Primers.

Genetic crosses were done using standard techniques (Weigel and Glazebrook 2006). Phire Plant Direct PCR kit (ThermoFisher Scientific F130) was used for genotyping with dilution protocol. Genotyping of the soq1-1 allele was done either by sequencing of a 800 bp PCR product amplified with primers MDB74 forward (TAGGTGTGCCTACCAGCGAG) (SEQ ID NO:6) and MDB72 reverse (TGAGCCACCAGTGAGAATGTC) (SEQ ID NO:7) surrounding the point mutation, position G372 to A in mutant, or by amplifying a 248 bp product with dCAPS primers (Neff et al., 2002) AM145 forward (GAAGTGGTTTCTTTTGTACAATTCTGCA) (SEQ ID NO:8) and AM146 reverse (CAATACGAATAGCGCACACG) (SEQ ID NO:9) that is digested by Pstl if wild-type allele. To genotype the chlKO T-DNA allele, AM164 forward (LP) (CCGCTTTGACATTTACATTACG) (SEQ ID NO:10) and AM165 reverse (RP) (TATAGCAATGTCGGCTCCAAC) (SEQ ID NO:11) were used with LBb1.3 to amplify a 569 bp product in wild-type (LP+RP), a 869 bp (with insert) in chlKO (LBb1.3+RP) or both in heterozygous individuals according to the Salk Institute Genomic Analysis Laboratory T-DNA primer design tool.

EMS Mutagenesis and Screening of Suppressor Mutants.

M2 seedlings were derived from mutagenesis of soq1 npq4 gl1 seeds with 0.24% (v/v) ethyl methane sulfonate (EMS). Suppressors of soq1 npq4 were screened based on their NPQ phenotype by chlorophyll fluorescence video imaging (Niyogi et al., 1998) using an Imaging-PAM Maxi (WALZ). For mutant screening, 60 to 80 seeds were plated per agar plate and 3 week-old seedlings were dark-acclimated for 20 min prior to measurement.

Mutation mapping and identification by whole genome sequencing. To identify the mutation of interest, the A205 mutant (soq1 npq4 gl1 chl-3) was crossed to the soq1 npq4 gl1 parental line, which was used for generation of the EMS population. Plants displaying the mutant phenotype (low NPQ) in the F2 generation were identified and pooled for DNA extraction. Genomic DNA was extracted from soq1 npq4 gl1×A205 F2 mutant plants (pool of 75 seedlings), soq1 npq4 gl1 (150 seedlings), and A252 M3 mutant pool (200 seedlings) using the Gentra Puregene kit (Qiagen). Genomic DNA was submitted to the Functional Genomics Laboratory (UC Berkeley) for preparation of the sequencing libraries, which were sequenced at the Vincent J Coates Genomics Sequencing Laboratory (UC Berkeley). The three samples were multiplexed and run with an unrelated sample in two lanes on an 11lumina HiSeq 2000/2500 to obtain 100 bp paired-end reads. The sequencing reads were mapped to the Col-0 reference genome (TAIR) and SNPs were detected using the CLC Genomics Workbench software. The SNPs present in the soq1 npq4 gl1 background were subtracted from those identified in the A205 mutant to identify SNPs likely to have been induced by this new round of EMS mutagenesis and therefore to be segregating in the mapping population. The SNPs were further filtered by coverage (between 20 and 200×), observed frequency (>25%), and mapping quality. The allelic frequency of each SNP in the pooled A205 mutant F2 was then plotted relative to the genomic position (FIGS. 9A to 9E) to identify the region showing linkage to the causative mutation. The set of genes containing an amino-acid changing mutation within this region for the A205 pool was then compared to the genes containing mutations in the A252 mutant.

Chlorophyll Fluorescence Measurement.

Chlorophyll fluorescence was measured at room temperature from attached, fully expanded rosette leaves or leaf discs of same area using a Dual-PAM-100 (Walz) fluorimeter. Plants were dark-acclimated for 20 min and NPQ was induced by 1200 μmol photons m⁻² s⁻¹ (red actinic light) for 10 min and relaxed in the dark for 10 min unless stated otherwise. Maximum fluorescence levels after dark-acclimation (Fₘ) and throughout measurement (Fₘ′) were recorded after applying a saturating pulse of light. NPQ was calculated as (Fₘ−Fₘ′)/Fₘ′. For the cold and high light treatment, leaf discs of same area were extracted from 4 different plant individuals of each genotype after 8 h and placed at room temperature for 40 min in the dark on a moist surface, initial fluorescence (F₀) and Fₘ was measured on each of these leaf discs (16 total) in a staggered order (e.g. Col-0, soq1, chlKO, soq1 chlKO leaf disc number 1, then Col-0, soq1, chlKO, soq1 chlKO leaf disc number 2, etc.). One-way ANOVA followed by Tukey's multiple comparisons test was performed using GraphPad Prism version 7.0a for Mac (GraphPad Software, La Jolla, Calif. USA).

Protein Extraction and Immunoblot Analysis.

Total cell extracts were isolated from same leaf area and solubilized in 200 mM dithiothreitol (DTT), 100 mM EDTA (initial pH 8.0), 120 mM Tris HCl (initial pH 6.8), 4% SDS and 12% sucrose at 100° C. for 10 min (adapted from (Tsugama et al., 2011)). Thylakoids were isolated as described (Casazza et al., 2001) and solubilized at 70° C. for 4 min in the same solubilization buffer as above with or without DTT. For immunoblots, samples were loaded by chlorophyll content (3.5 μg per lane) for thylakoids or leaf area for total cell extracts on a anyKD gel (BioRad), separated by SDS-PAGE, transferred in a semidry blotting apparatus at 0.8 mA cm⁻² for 1h to a PVDF membrane, blocked with 3% (w/v) nonfat dry milk, and incubated with the following antibodies. Rabbit-specific antibodies against a C-terminal peptide of SOQ1 (TVTPRAPDAGGLQLQGTR) (SEQ ID NO:12) were produced and purified by peptide affinity by ThermoFisher and used at a 1:2,000 dilution. Anti-CHL antibody against recombinant protein (Levesque-Tremblay et al., 2009) was provided by F. Ouellet (Université du Québec à Montréal) and used at a 1:2,000 dilution. PsbO antibody was obtained from Agrisera (AS06 142-33) and used at a 1:2,000 dilution. After incubation with HRP-conjugated secondary antibody, bands were detected by chemiluminescence with ECL substrate (GE Healthcare).

Pigment Extraction and Analysis.

HPLC analysis of carotenoids and chlorophylls was done as previously described (Müller-Moulé et al., 2002). Carotenoids were quantified using standard curves of purified pigments (VKI) and normalized to chlorophyll a. For the cold and high light treatment, pigments were extracted from the same leaf discs used for the fluorescence measurement (4 samples per genotype per time point).

### Example 2

### The Chloroplastic Lipocalin is Involved in a Sustained Photoprotective Mechanism Regulated by the Suppressor of Quenching 1 Protein in Arabidopsis thaliana

Overexpression of OTK1 Prevents CHL-Dependent Quenching from Occurring.

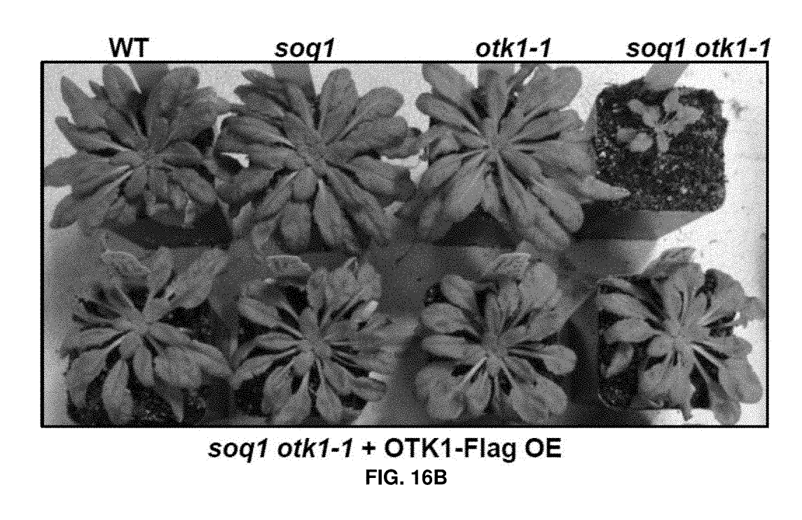

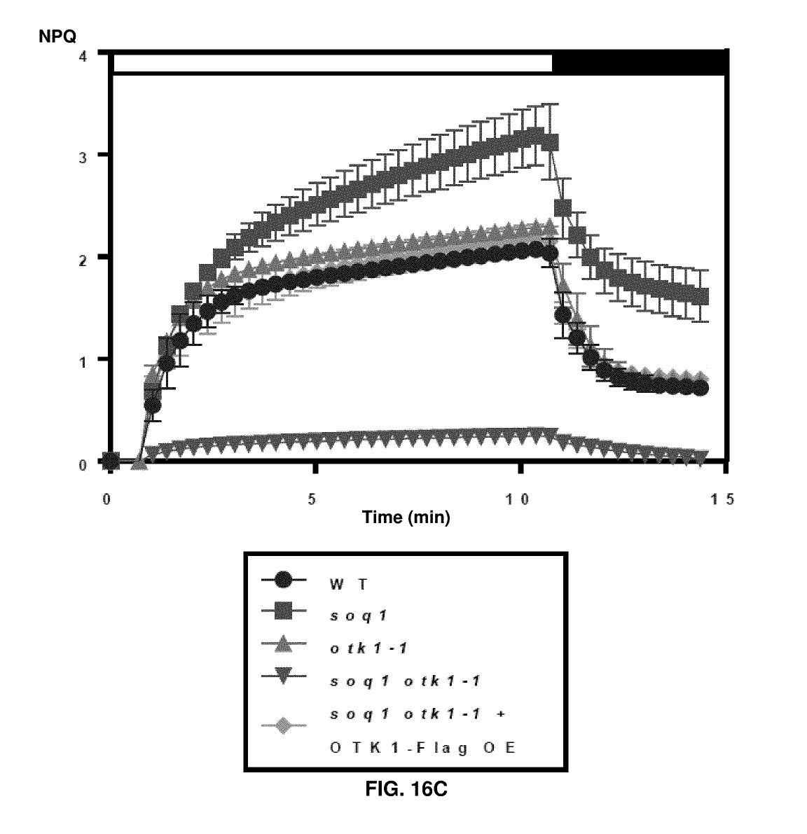

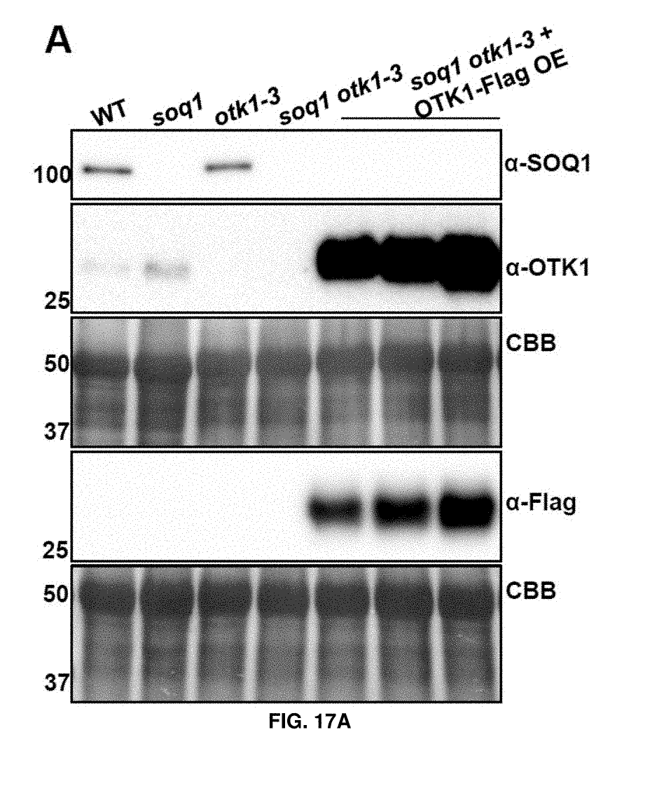

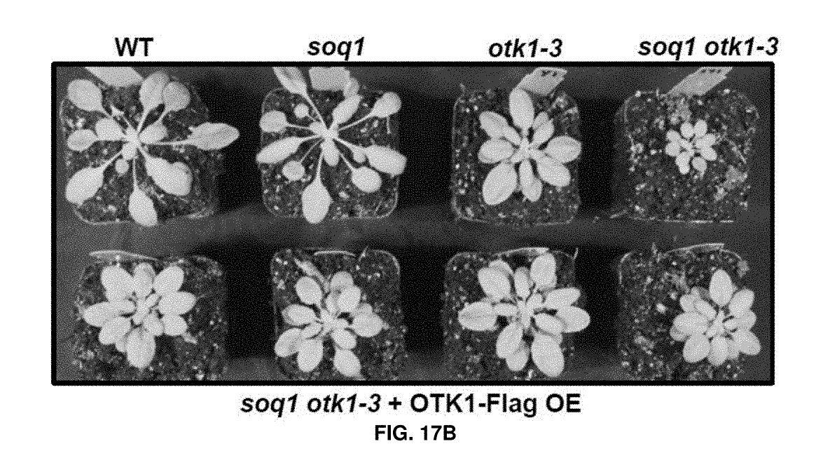

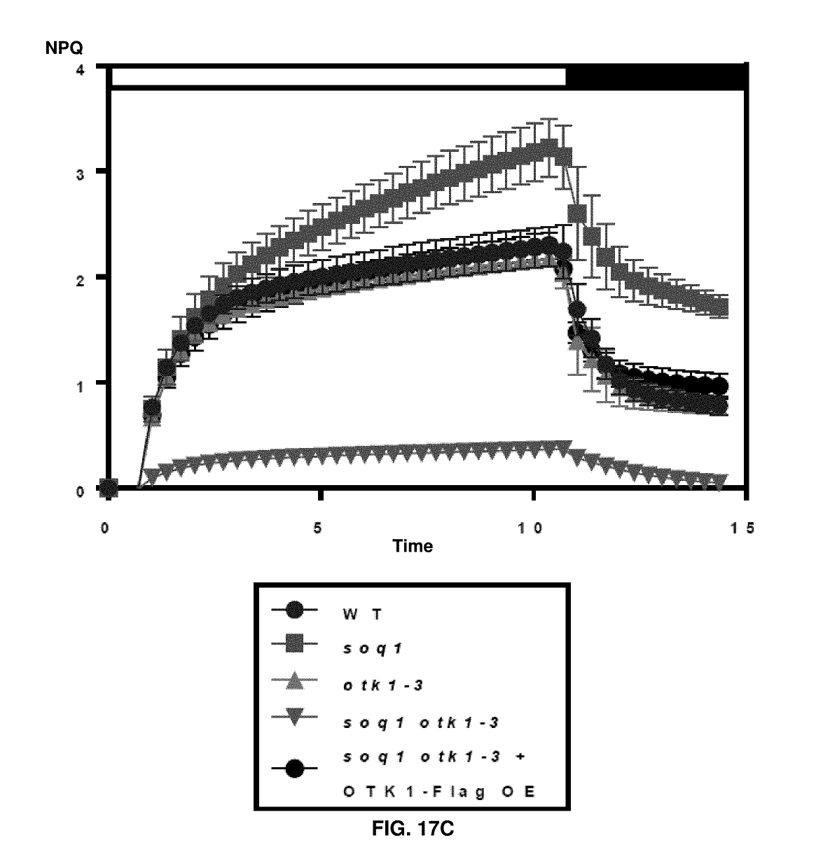

Constitutive quenching occurs in the absence of SOQ1 and OTK1, suggesting that OTK1 negatively regulates CHL-dependent quenching. However, the NPQ phenotype of the soq1 single mutant indicates that quenching can still occur in the presence of OTK1. The NPQ phenotype of soq1 led us to question whether the inhibiting function of OTK1 is dosage dependent. To test the dosage effect of OTK1, we overexpressed OTK1 in the soq1 otk1-1 mutant background (FIG. 16A). Overexpression of OTK1 returned growth (FIG. 16B), Fo, and Fm of soq1 otk1-1 to wild type levels. Surprisingly, overexpression restored NPQ to wild type levels and not to soq1 levels (FIG. 16C). To ensure this level of NPQ was not due to the residual OTK1-1 protein that accumulated in soq1 otk1-1, we overexpressed OTK1 in the soq1 otk1-3 mutant background. The NPQ phenotype of soq1 otk1-3+OTK1-Flag OE also reached wild type levels, confirming previous result (FIGS. 17A to 17C).

1. A method for improving photosynthesis in a plant cell or plant, comprising the reducing the expression of a Chloroplastic Lipocalin (CHL) gene in a plant cell or plant whereby the plant cell or plant, when cultured or grown under conditions suitable for photosynthesis, increases photosynthesis within the plant cell or plant.

2. The method of claim 1, wherein the reducing step comprises mutating the CHL gene in the plant cell or plant such that the mutated CHL gene has reduced or no biological activity, reduced transcription of the CHL gene, or the CHL gene is knocked-out, or silencing the expression of the CHL gene through an introduced iRNA or antisense RNA construct in the plant cell or plant that is specific for the CHL gene.

3. A method for improving photosynthesis in a plant, comprising the steps of eliminating a sustained photoprotective mechanism in a plant by mutating or silencing the Chloroplastic Lipocalin (CHL) gene whereby photosynthesis of the plants increases.

4. A polynucleotide encoding a mutant CHL protein, wherein the mutant CHL protein has reduced or no biological activity.

5. The polynucleotide of claim 4, wherein the mutant CHL protein is chl-2 (AtCHL-A255V).

6. The polynucleotide of claim 4 or 5, wherein an open reading frame (ORF) encoding the mutant CHL protein is operatively linked to a promoter capable of transcribing the ORF encoding the mutant CHL protein.

7. An expression cassette that incorporates the polynucleotide of any one of claims 4-6 and expresses a mutant CHL protein that has reduced or no biological activity.

8. A cell comprising the expression cassette of claim 7 in its genome.

9. A plant incorporating the cell of claim 5, whereby photosynthesis of the plant is improved or increased, and the photoprotective mechanisms are decreased or eliminated as compared to wild type.
